(19) United States
(12) Patent Application Publication
(10) Pub. No.: US 2022/0056407 A1
(43) Pub. Date: Feb. 24, 2022

(54) CELL

(71) Applicant: AUTOLUS LIMITED, London (GB)

(72) Inventors: Paul Smith, London (GB); Martin Pulé, London (GB); Simon Thomas, London (GB); Isaac Gannon, London (GB)

(21) Appl. No.: 17/312,755

(22) PCT Filed: Dec. 13, 2019
(86) PCT No.: PCT/GB2019/053538

§ 371 Date: Jun. 10, 2021

(30) Foreign Application Priority Data
Dec. 14, 2018 (GB) 1820443.8
Mar. 26, 2019 (GB) 1904160.7
May 21, 2019 (GB) 1907169.5

(51) Int. Cl. C12N 5/0783; A61K 35/17; C07K 14/725

(52) U.S. Cl. CPC C12N 5/0636; C07K 14/7051; A61K 35/17

(57) ABSTRACT

The present invention provides an engineered cell, such as a T-cell, which expresses a chimeric antigen receptor (CAR) or an engineered T-cell receptor (TCR) and one or more enzymes which, when secreted or expressed at the cell surface causes depletion of a molecule extracellular to the engineered cell; wherein said molecule is selected from: an amino acid; a nucleotide or nucleoside; or a lipid.

### FIELD OF THE INVENTION

The present invention relates to an engineered cell which co-expresses a chimeric antigen receptor (CAR) or engineered T-cell receptor (TCR) with one or more enzymes.

### BACKGROUND TO THE INVENTION

Adoptive immunotherapy involves the ex vivo generation of cancer-antigen specific cells and their administration to patients.

The native specificity of immune effector cells can be exploited in adoptive immunotherapy—for example during the generation of melanoma specific T-cells from expansion of tumour infiltrating lymphocytes in tumour resections. Otherwise a specificity can be grafted onto a cell (e.g. a T-cell) using genetic engineering. Two common methods for achieving this are using chimeric antigen receptors or transgenic T-cell receptors. Different kinds of immune effector cells can also be used. For example, alpha/beta T-cells, NK cells, gamma delta T-cells or macrophages can be used.

Adoptive immunotherapy has been successful in treating a number of lymphoid malignancies, such as B-cell Acute Lymphoblastic Leukaemia (B-ALL), Diffuse Large B-cell Lymphoma (DLBCL) and Multiple Myeloma (MM), however there has been relatively little success in the treatment of other cancers, particularly solid tumours.

Engineered cells face hostile microenvironments which can limit the effectiveness of adoptive immunotherapy. For example, the glycolytic metabolism of tumour cells renders the tumour microenvironment hypoxic, acidic, low in nutrients, and prone to oxidative stress, making it difficult for adoptive cells to survive and persist.

There is thus a need for alternative CAR treatment approaches which address the problems associated with survival, engraftment and proliferation of CAR-expressing cells in the hostile tumour microenvironment.

### DESCRIPTION OF THE FIGURES

FIG. 1—Schematic showing different generations of chimeric antigen receptors. The basic architecture of a canonical CAR is shown as well as different iterations of the three generations of this form of receptor.

FIG. 2—Schematic diagram illustrating the kynurenine pathway.

FIG. 3—Schematic diagram illustrating the adenosine pathway.

FIG. 4—Schematic diagram illustrating the arginine pathway. OTC=orinithine transcarbamylase, Uniprot P00480; ASS=argininosuccinate synthetase, Uniprot P00966; ASL=argininosuccinate lyase, Uniprot P04424

FIG. 5—The arginine biosynthetic pathway in bacteria. ArgA: Uniprot POA6C5; ArgB Uniprot POA6C8; ArgC: Uniprot P11446; ArgD: Uniprot P18335: ArgE: Uniprot P23908; ArgF: Uniprot P06960; ArgH: Uniprot P11447; Argl: Uniprot P04391. Citrulline may be given as a dietary supplement. Citrulline import is mediated by the L-type amino acid transporter (LAT1). Citrulline may be processed to arginine by the expression of ArgG and ArgH.

FIG. 6—Valine biosynthesis. E. coli pathway enzymes: ilvl: Uniprot P00893; ilvC: Uniprot P05793; ilvD: Uniprot P05791; ilv: Uniprot POAB80.

FIG. 7A—Homoserine biosynthesis. E. coli pathway enzymes: ThrA: Uniprot P00561; asd: Uniprot POA9Q9. Steps 1 and 3 can be encoded by a fused aspartate kinase/homoserine dehydrogenase 1 enzyme. FIG. 7B—Threonine biosynthesis. E. coli pathway enzymes ThrB: Uniprot P00547; ThrC: Uniprot P00934.

FIG. 8—Methionine biosynthesis. Steps 1 to 3 are from homoserine biosynthesis pathway (FIG. 7A). MetA: Uniprot P07623; MetB: Uniprot P00935; MetC: Uniprot P06721. Final step from homocysteine can be catalysed by H. sapiens Methionine synthase (MTR, Uniprot Q99707) or E. coli metH: Uniprot P13009. Homocysteine can be given as a dietary supplement

FIG. 9—Lysine biosynthesis. Steps 1 to 2 are from homoserine biosynthesis pathway (FIG. 7A). dapA (E. coli): Uniprot POA6L2; dapB (E. coli): Uniprot P04036; ddh (Corynebacterium glutamicum): Uniprot P04964; lysA (E. coli): Uniprot P00861.

FIG. 10—Tryptophan biosynthesis. E. coli enzymes: trpA: Uniprot P0A877, trpA activity may be increased by trpB: Uniprot P0A879; trp C: Uniprot P00909 (single fused enzyme catalyses 2 steps); trpD: Uniprot P00904. Anthranilate and 5-phospho-ribose 1-diphosphate are produced by human metabolism. Anthranilate can also be given as a dietary supplement.

FIG. 11—Schematic diagram of the tumour microenvironment.

FIG. 12—Depletion of methionine in culture medium following culture of T cells expressing methioninase or methionine gamma lyase enzymes.

SupT1 cells expressing Methioninase (Pseudomonas putida: Uniprot P13254), Methionine gamma lyase (Kluyveromyces lactis: Uniprot Q6CKK3), or Methionine gamma lyase (Kluyveromyces lactis: Uniprot Q6CKK4) were cultured for 24 or 96 hours and methionine in the culture medium was assayed. Non-transduced (NT) cells were used as a negative control and recombinant methioninase from P. putida was added to culture medium as a positive control.

FIG. 13—Depletion of phenylalanine in culture medium following culture of T cells expressing phenylalanine/tyrosine ammonia lyase (PTAL).

Retroviral constructs encoding genes for Phenylalanine/tyrosine ammonia lyase (PTAL) were transduced into SupT1 T cell line. Expression of encoded genes was analysed by expression of V5 Tag expression. Cells were plated at 100,000 cells/ml for 24, 48, 72 or 144 hours and levels of phenyalanine in culture medium was assessed by Phenylalanine assay kit (Biovision). Non-transduced (NT) cells were used as a negative control.

### SUMMARY OF ASPECTS OF THE INVENTION

The present inventors have found that it is possible to optimise the function of CAR-expressing or TCR-expressing cells by engineering the cell to express one or more enzymes which, when secreted or expressed at the cell surface, causes depletion of a molecule extracellular to the engineered cell which is:
(i) required by a tumour cell for survival, proliferation, metastasis or chemoresistance, and/or
(ii) detrimental to the survival, proliferation or activity of the engineered cell.

This technology has many applications, including modulating the microenvironment in favour of the immune response, which in turn helps to optimise adoptive immunotherapy.

In a first aspect, the present invention provides an engineered cell which expresses a chimeric antigen receptor (CAR) or engineered T-cell receptor (TCR) and one or more enzymes which, when secreted or expressed at the cell surface, causes depletion of a molecule extracellular to the engineered cell which is:
(i) required by a tumour cell for survival, proliferation, metastasis or chemoresistance, and/or
(ii) detrimental to the survival, proliferation or activity of the T-cell.

The cell may be a T cell.

The engineered cell may express one or more enzymes which, when secreted or expressed at the cell surface, causes depletion of a molecule extracellular to the engineered cell; wherein said molecule is selected from: an amino acid; a nucleotide or nucleoside; or a lipid, or a derivative thereof.

The molecule may be a derivative or a precursor of an amino acid; a nucleotide or nucleoside; or a lipid. For example the molecule may be an amino acid derivative such as an amino acid metabolite.

The molecule may be an amino acid such as: isoleucine, leucine, lysine, methionine, phenylalanine, threonine, tryptophan valine serine, glycine, cysteine or proline.

In order to deplete arginine, the engineered cell may secrete or express arginase, arginine deaminase and/or arginine decarboxylase.

In order to deplete phenylalanine, the engineered cell may secrete or express phenyalanine-ammonia lyase.

The molecule may be a derivative of an amino acid, such as an amino acid metabolite. The molecule may be a tryptophan metabolite, such a kynurenine. In order to deplete kynurenine, the engineered cell may secrete or express kynureninase.

The molecule may be a nucleotide or nucleoside such as adenosine.

In order to deplete adenosine, the engineered cell may secrete or express adenosine deaminase or AMP deaminase.

The molecule may be a lipid, such as a lipid selected from the following group: Prostaglandin E2 (PGE2), Sphingosine-1-phosphate (S-1-P) and Lysophosphatidic acid (LPA). Suitably, the lipid may be Prostaglandin E2 (PGE2). Suitably, the lipid may be Sphingosine-1-phosphate (S-1-P). Suitably, the lipid may be Lysophosphatidic acid (LPA).

The enzyme(s) may convert the molecule into a product which is detrimental to the survival or proliferation of a tumour cell and/or which promotes the proliferation and/or activity of the T-cell.

In this respect, the product may be agmatine, tryptamine, dimethyltryptamine, tyramine, histamine, phenylethylamine or cinnamic acid.

The present invention also provides a cell which is engineered to survive in the absence of a molecule in the extracellular environment. The molecule may be required by a tumour cell for survival, proliferation, metastasis or chemoresistance.

One way of achieving this is to engineer the cell to synthesise the molecule or a precursor thereof intracellularly.

The molecule may be an amino acid; a nucleotide or nucleoside; or a lipid, or a derivative thereof. The molecule may be an amino acid such as an essential amino acid.

For example the cell may be engineered to synthesise isoleucine, leucine, lysine, methionine, arginine, phenylalanine, threonine, tryptophan or valine intracellularly.

The present invention also provides a cell which expresses or over-expresses one or more amino acid transporter(s). The cell may be engineered to comprise a polynucleotide encoding an amino acid transporter. The amino acid transporter may be selected from the list of amino acid transporters given in Table 1 of Hyde et al (2003) 373:1-18. The amino acid transporter may be L-type amino acid transporter 1 (LAT1).

In a second aspect, the present invention provides a nucleic acid construct which comprises: (i) a first polynucleotide which encodes an enzyme as defined herein; and (ii) a second polynucleotide which encodes a chimeric antigen receptor (CAR) or a transgenic T-cell receptor (TCR).

The first and second polynucleotides of the nucleic acid construct may be separated by a co-expression site.

In a third aspect, the present invention provides a kit of polynucleotides comprising: (i) a first polynucleotide which encodes an enzyme as defined herein; and (ii) a second polynucleotide which encodes a chimeric antigen receptor (CAR) or a transgenic T-cell receptor (TCR).

In a fourth aspect, the present invention provides a vector which comprises a nucleic acid construct according to the present invention.

In fifth aspect, the present invention provides a kit of vectors which comprises: (i) a first vector which comprises a polynucleotide which encodes an enzyme as defined herein; and (ii) a second vector which comprises a polynucleotide which encodes a chimeric antigen receptor (CAR) or a transgenic T-cell receptor (TCR).

In a sixth aspect the present invention provides a pharmaceutical composition which comprises a cell according to the first aspect of the invention.

In a seventh aspect, there is provided a pharmaceutical composition according to the sixth aspect of the invention, for use in treating a disease.

In an eighth aspect, there is provided a method for treating a disease, which comprises the step of administering a pharmaceutical composition according to the sixth aspect of the invention to a subject in need thereof.

The method may comprise the following steps:
(i) isolation of a cell containing sample;
(ii) introducing a nucleic acid construct according to second aspect of the invention, a kit of polynucleotides according to the third aspect of the invention, a vector according to the fourth aspect of the invention; or a kit of vectors according to the fifth aspect of the invention to cells from the cell-containing sample; and
(iii) administering the cells from (ii) to a subject.

In another embodiment, there is provided the use of a cell according to the first aspect of the invention in the manufacture of a medicament for the treatment of a disease.

The disease may be cancer, such as a solid cancer

In a further embodiment, there is provided a method for making a cell according to the present invention, which comprises the step of introducing: a nucleic acid construct according to second aspect of the invention; a kit of polynucleotides according to the third aspect of the invention, a vector according to the fourth aspect of the invention; or a kit of vectors according to the fifth aspect of the invention into a cell ex vivo.

In a tumour microenvironment, tumour cells and associated cells such as carcinoma-associated fibroblasts (CAF), myeloid-derived suppressor cells (MDSC) and tumour-associated macrophages (TAM) are in competition with immune cells for nutrients. The immune microenvironment contains small molecule metabolites and nutrients and altering the balance of these molecules can shift the microenvironment either in favour or tumour survival or in favour of progression of the immune response.

The present invention provides engineered cells which have an in-built capacity to skew the microenvironment in favour of the immune response, for example in favour of a T cell response involving adoptively transferred T cells.

The microenvironment may be skewed in favour of the immune response by depleting a molecule required by a tumour cell for survival, proliferation, metastasis or chemoresistance. The cells of the immune response, such as CAR-T cells may have a lower dependency than the tumour cells for the molecule either naturally, or because they are engineered either to make it or survive/proliferate without it.

Alternatively the microenvironment may be skewed in favour of the immune response by depleting a molecule detrimental to the survival, proliferation or activity of the T-cell.

The present invention therefore provides cells with an in-built mechanism to modulate the microenvironment and alter the balance in favour of the immune response. Such cells have an enhanced ability to survive in the tumour microenvironment and successfully out-compete tumour cells.

Further Aspects

Further aspects of the invention are summarised in the following numbered paragraphs.

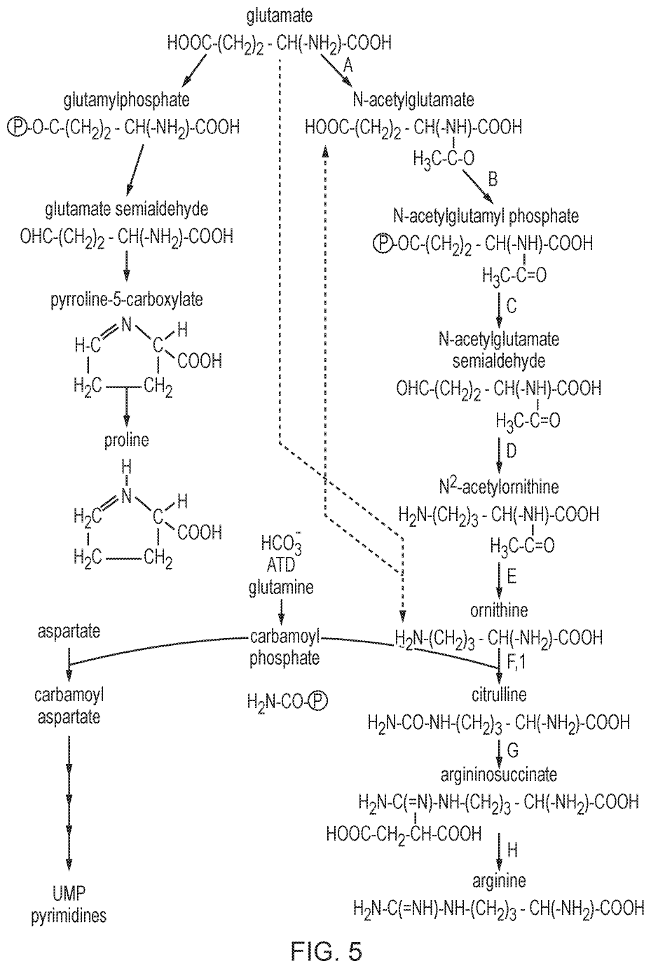

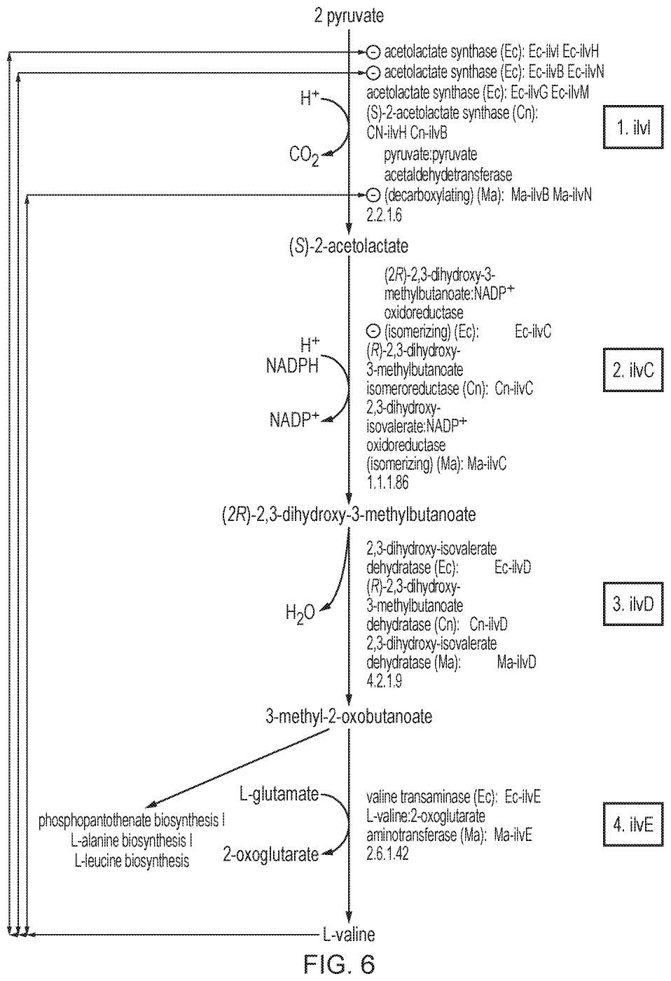

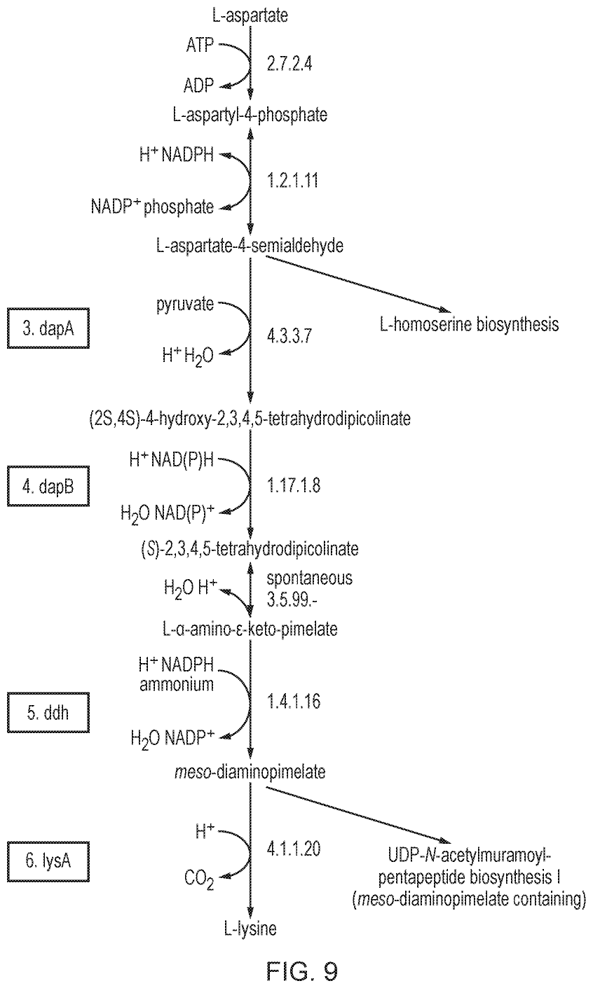

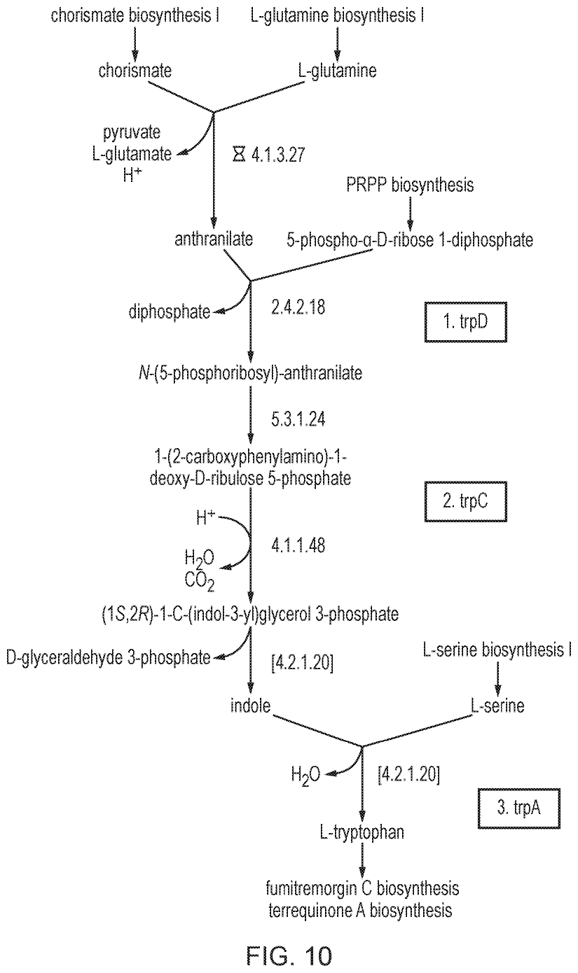

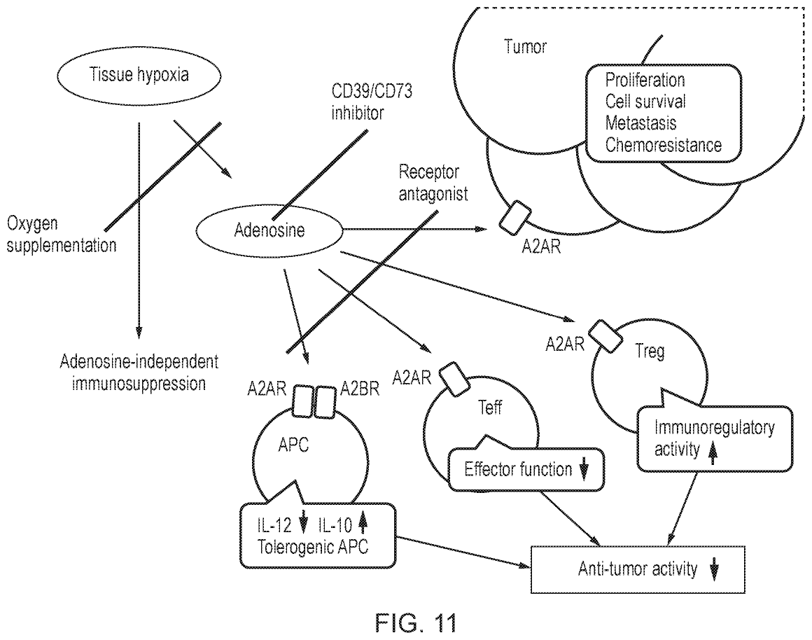

1. A kit of polynucleotides comprising: (i) a first polynucleotide which encodes one or more enzymes involved in the intracellular synthesis of a molecule; and (ii) a second polynucleotide which encodes a chimeric antigen receptor (CAR) or a transgenic T-cell receptor (TCR).
2. A kit of vectors which comprises: (i) a first vector comprising a polynucleotide which encodes one or more enzymes involved in the intracellular synthesis of a molecule; and (ii) a second vector comprising a polynucleotide which encodes a chimeric antigen receptor (CAR) or a transgenic T-cell receptor (TCR).
3. A kit according to paragraph 1 or 2, wherein the molecule is required by a tumour cell for survival, proliferation, metastasis or chemoresistance.
4. A kit according to any preceding paragraph, wherein the molecule is an amino acid; a nucleotide or nucleoside; or a lipid, or a derivative thereof.
5. A kit according to any preceding paragraph, wherein the molecule is an amino acid.
6. A kit according to paragraph 5, wherein the molecule is an essential amino acid.
7. A kit according to paragraph 5 or 6, wherein the molecule is isoleucine, leucine, lysine, methionine, arginine, phenylalanine, threonine, tryptophan or valine.
8. A kit according to any preceding paragraph, wherein the one or more enzyme(s) is/are a bacterial enzyme(s).
9. A kit according to any preceding paragraph, wherein the one or more enzyme(s) is/are one or more of the enzymes involved in the biosynthetic pathways shown in FIGS. 5 to 10.
10. A kit according to paragraph 7, wherein the molecule is arginine.
11. A kit according to paragraph 10, wherein the one or more enzymes are selected from: orinithine transcarbamylase (OTC), argininosuccinate synthetase 1 (ASS1), argininosuccinate lyase 1 (ASL1)
12. A kit according to paragraph 10 or 11 which also comprises a nucleic acid sequence encoding L-type amino acid transporter (LAT1).
13. A cell which expresses a chimeric antigen receptor (CAR) or a transgenic T-cell receptor (TCR) and which is engineered to survive in the absence of the molecule in the extracellular environment.
14. A cell according to paragraph 13, which is engineered to:
synthesise the molecule or a precursor thereof intracellularly;
inhibit the intracellular breakdown of the targeted products; and/or
increase the efficiency of the import of the molecule or precursor thereof.
15. A cell according to paragraph 14, engineered to express one or more enzymes involved in the intracellular synthesis of the molecule.
16. A cell according to any of paragraphs 13 to 15, wherein the molecule is required by a tumour cell for survival, proliferation, metastasis or chemoresistance.
17. A cell according to any of paragraphs 13 to 16, wherein the molecule is an amino acid; a nucleotide or nucleoside; or a lipid, or a derivative thereof.
18. A cell according to any of paragraphs 13 to 17, wherein the molecule is an amino acid.
19. A cell according to paragraph 18, wherein the molecule is an essential amino acid.
20. A cell according to paragraph 18 or 19, wherein the molecule is isoleucine, leucine, lysine, methionine, arginine, phenylalanine, threonine, tryptophan or valine.
21. A cell according to paragraph 15, wherein the one or more enzyme(s) is/are a bacterial enzyme(s).
22. A cell according to paragraph 15, wherein the one or more enzyme(s) is/are one or more of the enzymes involved in the biosynthetic pathways shown in FIGS. 5 to 10.
23. A cell according to paragraph 20, wherein the molecule is arginine.
24. A cell according to paragraph 23, engineered to express one or more of the following enzymes: orinithine transcarbamylase (OTC), argininosuccinate synthetase 1 (ASS1), argininosuccinate lyase 1 (ASL1)
25. A cell according to paragraph 23 or 24, which comprises a nucleic acid sequence encoding L-type amino acid transporter (LAT1).
26. A cell according to paragraph 20, which is engineered to synthesise tryptophan.
27. A cell according to paragraph 26, wherein the molecule is a tryptophan metabolite.
28. A cell according to paragraph 27, wherein the molecule is kynurenine.
29. A cell according to paragraph 27 or 28 wherein the cell secretes kynureninase or expresses kynureninase at its cell surface.
30. A nucleic acid construct which comprises (i) a polynucleotide which encodes one or more enzymes involved in the intracellular synthesis of a molecule; and (ii) a polynucleotide which encodes a chimeric antigen receptor (CAR) or a transgenic T-cell receptor (TCR).
31. A vector comprising a nucleic acid construct according to paragraph 30.
31. A method for making a cell according to any of paragraphs 13 to 19 which comprises the step of introducing a nucleic acid construct according to paragraph 30, a vector according to paragraph 31, or a kit of polynucleotides or vectors according to any of paragraphs 1 to 12 into the cell ex vivo.
32. A pharmaceutical composition which comprises a plurality of cells according to any of paragraphs 13 to 19.
33. A pharmaceutical composition according to paragraph 32, for use in treating a disease.
34. A method for treating a disease, which comprises the step of administering a pharmaceutical composition according to paragraph 33 to a subject in need thereof.
35. A method according to paragraph 34, which comprises the following steps:
(i) isolation of a cell containing sample;
(ii) introducing a nucleic acid construct according to paragraph 30, a vector according to paragraph 31, or a kit of polynucleotides or vectors according to any of paragraphs 1 to 12 to the cell ex vivo; and
(iii) administering the cells from (ii) to a subject.
36. A method according to paragraph 34 or 35 which comprises the following steps:
(i) administering a pharmaceutical composition to the subject wherein the pharmaceutical composition comprises cells capable of synthesizing the molecule from a precursor; and
(ii) administering the precursor to the subject.
37. A method according to paragraph 36, wherein the molecule is arginine and the precursor is citrulline.
38. A method according to claim 33, wherein the cells are engineered to express L-type amino acid transporter (LAT1).
39. The use of a cell according to any of paragraphs 13 to 19 in the manufacture of a medicament for the treatment of a disease.
40. The pharmaceutical composition for use according to paragraph 33, the method according to any of paragraphs 34 to 38, or the use according to paragraph 39, wherein the disease is cancer.

### DETAILED DESCRIPTION

The present invention provides an engineered cell which expresses a chimeric antigen receptor (CAR) or engineered T-cell receptor (TCR) together with one or more enzymes.

Enzyme

As used herein, “enzyme” refers to the biological catalyst which the cell has been engineered to express at the cell surface, or to secrete, which is capable of causing depletion of a molecule extracellular to the engineered cell according to the present invention.

Suitably, the enzyme may directly cause depletion of said molecule. In other words, the enzyme may act directly on said molecule i.e. the depletion of said molecule is not an indirect effect of the enzyme. Said molecule is selected from: an amino acid; a nucleotide or nucleoside; or a lipid.

The molecule may be required by a tumour cell for survival.

As used herein “required by a tumour cell” means that in the absence of the molecule, the survival, proliferation, metastasis and/or chemoresistance of the tumour cell is compromised, reduced or completely abolished.

Suitably, in the absence of a required molecule, the survival, proliferation metastasis and/or chemoresistance of the tumour cell may be reduced by at least 5%, at least 10%, at least 20%, at least 30%, at least 40%, at least 50%, at least 60%, at least 70%, at least 80%, at least 90%, at least 95% or at least 99%.

The molecule may be detrimental to the survival of the engineered cell (such as engineered T-cell).

As used herein “molecule which is detrimental to” means that in the presence of the molecule, the survival, proliferation or activity of the engineered cell (such as an engineered T-cell) is compromised, reduced or completely abolished.

Suitably, in the presence of the detrimental molecule, the survival, proliferation and/or activity of the engineered cell (such as an engineered T-cell) may be reduced by at least 5%, at least 10%, at least 20%, at least 30%, at least 40%, at least 50%, at least 60%, at least 70%, at least 80%, at least 90%, at least 95% or at least 99%.

Cell survival (such as tumour cell survival or T-cell survival) may be measured by methods known in the art.

Suitable methods include measuring the size of the cell population (e.g. by counting cells) or by measuring the number of viable cells. The number of viable cells can be determined by measuring apoptosis by 7AAD and Annexin V staining using flow cytometry. Other suitable methods include MTT assays, which assess cell metabolic activity via NAD(P)H-dependent cellular oxidoreductase enzymes. These enzymes reduce the tetrazolium dye MTT 3-(4,5-dimethylthiazol-2-yl)-2,5-diphenyltetrazolium bromide to its insoluble formazan, which has a purple colour.

The molecule may be required by a tumour cell for proliferation. The molecule may be detrimental to the proliferation of the engineered cell (such as engineered T-cell).

Cell proliferation may be measured by methods known in the art. Suitable methods include measuring the size of the cell population (e.g. by counting cells using a marker specific for the cell population, i.e. a tumour specific marker or an engineered cell specific marker, such as a CAR or transgenic TCR) or by performing cell cycle analysis using 5-bromo-2′-deoxyuridine (BrdU) which becomes incorporated into newly made DNA and propidium iodide (PI) and analysing by flow cytometry in combination with a cell population specific marker. Other suitable methods for measuring proliferation include MTT assays as described above.

The “activity” of an engineered cell may relate to its ability to engraft in a microenvironment, or to its ability to function as a CAR or transgenic TCR i.e. to bind to target antigen, activate, proliferate, cause cytotoxicity and/or secrete cytokines.

Signal Peptide

In one embodiment, the one or more enzymes as described herein require access to molecules extracellular to the engineered cell in order to cause depletion of said molecule.

In one embodiment the enzyme is capable of being secreted from the engineered cell of the invention. In one embodiment, the one or more enzymes are secreted from the engineered cell.

In another embodiment, the enzyme is capable of being expressed at (or on) the surface of the cell. In one embodiment, the one or more enzymes are expressed at (or on) the cell surface.

Suitably, the enzyme is expressed at the surface of the cell facing the extracellular space. Suitably, the active site of the enzyme may be extracellular.

The classical protein secretion pathway is through the endoplasmic reticulum (ER). The enzyme described herein may comprise a signal sequence so that when the proteins are expressed inside a cell, the nascent protein is directed to the ER.

The term “signal peptide” is synonymous with “signal sequence”.

A signal peptide is a short peptide, commonly 5-30 amino acids long, typically present at the N-terminus of the majority of newly synthesized proteins that are destined towards the secretory pathway. These proteins include those that reside either inside certain organelles (for example, the endoplasmic reticulum, Golgi or endosomes), are secreted from the cell, and transmembrane proteins.

Signal peptides commonly contain a core sequence which is a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide is commonly positioned at the amino terminus of the molecule, although some carboxy-terminal signal peptides are known.

Signal sequences typically have a tripartite structure, consisting of a hydrophobic core region (h-region) flanked by an n- and c-region. The latter contains the signal peptidase (SPase) consensus cleavage site. Usually, signal sequences are cleaved off co-translationally, the resulting cleaved signal sequences are termed signal peptides.

Signal sequences can be detected or predicted using software techniques (see for example, http://www.predisi.de/).

A large number of signal sequences are known, and are available in databases. For example, http://www.signalpeptide.de lists 2109 confirmed mammalian signal peptides in its database.

In one embodiment, the enzyme may be operably linked to a signal peptide which enables translocation of the enzyme into the endoplasmic reticulum (ER). The enzyme may be engineered to be operably linked to a signal peptide which enables translocation of the protein into the ER.

Suitably, the enzyme may operably linked to a signal peptide which is not normally operably linked to in nature. Suitably, the combination of the enzyme and the signal peptide may be synthetic (e.g. not found in nature).

In some embodiments an altered signal peptide (such as a less efficient signal peptide or a more efficient signal peptide) may be used. The use of an altered signal peptide may allow the system to be tuned according to clinical need.

Suitably, the signal peptide may be derived from human interleukin 2 (IL-2). An example of the sequence of human IL-2 is provided by UniProtKB Accession No: P60568: MYRMQLLSCIALSLALVTNSAPTSSSTKKTQLQLEHLLLDLQMILNGINNYKNPKLTRMLTFKF YMPKKATELKHLQCLEEELKPLEEVLNLAQSKNFHLRPRDLISNINVIVLELKGSETTFMCEYA DETATIVEFLNRWITFCQSIIS (SEQ ID NO: 1; UniProtKB Accession No: P60568).

In another embodiment, the enzyme may be operably linked to a signal peptide which enables translocation of the enzyme into the ER. Suitably, the enzyme may be operably linked to a signal peptide which it is normally operably linked to in nature. Suitably, the enzyme may comprise a wild-type signal peptide, e.g. the combination of the protein and signal peptide is naturally occurring.

In some embodiments, the enzyme is a membrane protein.

A “membrane protein” as used herein means a protein which comprises a membrane tethering component which acts as an anchor, tethering the protein to the cell membrane.

The membrane tethering component may comprise a membrane localisation domain. This may be any sequence which causes the protein to be attached to or held in a position proximal to the plasma membrane.

The membrane localisation domain may be or comprise a sequence which causes the nascent polypeptide to be attached initially to the ER membrane. As membrane material “flows” from the ER to the Golgi and finally to the plasma membrane, the protein remains associated with the membrane at the end of the synthesis/translocation process.

The membrane localisation domain may, for example, comprise a transmembrane domain or transmembrane sequence, a stop transfer sequence, a GPI anchor or a myristoylation/prenylation/palmitoylation site.

Alternatively the membrane localisation domain may direct the membrane-tethering component to a protein or other entity which is located at the cell membrane, for example by binding the membrane-proximal entity. The membrane tethering component may, for example, comprise a domain which binds a molecule which is involved in the immune synapse, such as TCR/CD3, CD4 or CD8.

Myristoylation is a lipidation modification where a myristoyl group, derived from myristic acid, is covalently attached by an amide bond to the alpha-amino group of an N-terminal glycine residue. Myristic acid is a 14-carbon saturated fatty acid also known as n-Tetradecanoic acid. The modification can be added either co-translationally or post-translationally. N-myristoyltransferase (NMT) catalyzes the myristic acid addition reaction in the cytoplasm of cells. Myristoylation causes membrane targeting of the protein to which it is attached, as the hydrophobic myristoyl group interacts with the phospholipids in the cell membrane.

The membrane tethering component of the present invention may comprise a sequence capable of being myristoylated by a NMT enzyme. The membrane tethering component of cell of the present invention may comprise a myristoyl group when expressed in a cell.

The membrane tethering component may comprise a consensus sequence such as: NH2-G1-X2-X3-X4-S5-X6-X7-X8 which is recognised by NMT enzymes.

Palmitoylation is the covalent attachment of fatty acids, such as palmitic acid, to cysteine and less frequently to serine and threonine residues of proteins. Palmitoylation enhances the hydrophobicity of proteins and can be used to induce membrane association. In contrast to prenylation and myristoylation, palmitoylation is usually reversible (because the bond between palmitic acid and protein is often a thioester bond). The reverse reaction is catalysed by palmitoyl protein thioesterases.

In signal transduction via G protein, palmitoylation of the a subunit, prenylation of the γ subunit, and myristoylation is involved in tethering the G protein to the inner surface of the plasma membrane so that the G protein can interact with its receptor.

The membrane tethering component may comprise a sequence capable of being palmitoylated. The membrane tethering component may comprise additional fatty acids when expressed in a cell which causes membrane localisation.

Prenylation (also known as isoprenylation or lipidation) is the addition of hydrophobic molecules to a protein or chemical compound. Prenyl groups (3-methyl-but-2-en-1-yl) facilitate attachment to cell membranes, similar to lipid anchors like the GPI anchor.

Protein prenylation involves the transfer of either a farnesyl or a geranyl-geranyl moiety to C-terminal cysteine(s) of the target protein. There are three enzymes that carry out prenylation in the cell, farnesyl transferase, Caax protease and geranylgeranyl transferase I.

The membrane tethering component may comprise a sequence capable of being prenylated. The membrane-tethering component may comprise one or more prenyl groups when expressed in a cell which causes membrane localisation.

A “transmembrane domain” as used herein is the sequence of a protein which spans the membrane. A transmembrane domain may be any protein structure which is thermodynamically stable in a membrane. This is typically an alpha helix comprising of several hydrophobic residues. The transmembrane domain of any transmembrane protein can be used to supply the transmembrane portion of a membrane protein according to the present invention.

The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components).

The enzyme may be a membrane protein (e.g. a protein comprising a membrane tethering component, a protein comprising a transmembrane domain). Suitably, the enzyme may be any type of membrane protein including without limitation: Types I, II and II (single pass molecules) and type IV (multiple-pass molecules) membrane proteins.

In some embodiments, the enzyme is a membrane protein and is anchored to the lipid membrane with a stop-transfer anchor sequence. In other embodiments, the enzyme is a membrane protein and is anchored to the lipid membrane with a signal-anchor sequence. In other embodiments, the enzyme is a membrane protein and its N-terminal domain is targeted to the cytosol. In a further embodiment, the enzyme is a membrane protein and its N-terminal domain is targeted to the lumen.

Molecule

As used here in, “depletion” means that the amount or concentration of the molecule extracellular to the engineered cell is reduced or eliminated completely by the one or more enzymes secreted or expressed at the surface of the engineered cell according to the present invention.

Suitably, the amount or concentration of the molecule extracellular to the engineered cell may be reduced by at least 5%, at least 10%, at least 20%, at least 30%, at least 40%, at least 50%, at least 60%, at least 70%, at least 80%, at least 90%, at least 95% or at least 99% by expression of the one or more enzymes at the cell surface of the engineered cell according to the present invention, or by secretion of the one or more enzymes from the engineered cell according to the present invention.

Suitably, the amount or concentration of the molecule extracellular to the engineered cell may be completely eliminated by expression of the one or more enzymes at the cell surface of the engineered cell according to the present invention, or by secretion of the one or more enzymes from the engineered cell according to the present invention.

The amount or concentration of the molecule extracellular to the engineered cell may be measured using any suitable method known in the art. For example, the concentration of the molecule may be determined by ELISA, for example adenosine, arginine and/or phenylalanine in tissue supernatants, may be measured by ELISA. Other methods include HPLC or liquid chromatography-mass spectrometry (LC-MS).

As used herein “a molecule extracellular to the engineered cell” means that the molecule is present outside of the engineered cell.

Suitably, the molecule may be present in the microenvironment e.g. a tumour microenvironment in the context of cancer.

Targeting Amino Acid Metabolism

Cancer cells undergo metabolic reprograming during proliferation to support their increased biosynthetic and energy demands. To meet these demands, cancer cells are thought to require a continuous supply of nutrients to maintain abnormal growth and rapid division. Amino acids are thought to be immunosuppressive in the tumour environment.

In one embodiment, the present invention provides an engineered cell for targeted cancer therapy by targeting amino acid metabolism, for example by depleting nutrients e.g. amino acids needed for tumour cell growth and division or depleting metabolites of biosynthetic pathways e.g. targeting arginine and tryptophan metabolism.

The present invention provides an engineered cell, such as an engineered T-cell, which expresses a chimeric antigen receptor (CAR) or engineered T-cell receptor (TCR) and one or more enzymes which, when secreted or expressed at the cell surface, causes depletion of an amino acid or derivative thereof.

In one aspect, the amino acid may be selected from: isoleucine, leucine, lysine, methionine, phenylalanine, threonine, tryptophan and valine. Suitably, the amino acid may be isoleucine. Suitably, the amino acid may be leucine. Suitably, the amino acid may be lysine. Suitably, the amino acid may be methionine. Suitably, the amino acid may be phenylalanine. Suitably, the amino acid may be threonine. Suitably, the amino acid may be tryptophan. Suitably, the amino acid may be valine.

In another aspect, the amino acid may be selected from arginine, serine, glycine, cysteine, and proline. Suitably, the amino acid may be arginine. Suitably, the amino acid may be serine. Suitably, the amino acid may be glycine. Suitably, the amino acid may be cysteine. Suitably, the amino acid may be proline.

Suitably, the amino acid may be an essential amino acid.

As used herein “essential amino acid” refers to an amino acid which cannot be synthesized de novo by the cell or organism.

In another embodiment, the amino acid may be a conditionally essential amino acid for a tumour.

As used herein “conditionally essential amino acid of a tumour” refers to an amino acid whose depletion restricts tumour growth and/or survival.

As many tumours require an increase in nutrients to maintain abnormal growth and division, a lack of even non-essential amino acids may become limiting for tumour growth.

A conditionally essential amino acid may be selected from: arginine, serine, glycine, cysteine, and proline.

Arginine

Arginine is a conditionally essential amino acid for several tumours. The arginine pathway is shown schematically in FIG. 4. Arginase degrades to urea and ornithine. Arginine deiminase degrades to ammonia and citrulline. Arginine decarboxylase degrades arginine to agmatine. Agmatine inhibits proliferation of tumour cells by modulation of polyamine metabolism.

In arginine synthesis, orinithine transcarbamylase (OTC) catalyses the transfer of the carbamoyl moiety of carbamoylphosphate to the 5-amino group of ornithine, forming citrulline (FIG. 4). The rate limiting step in arginine synthesis is the conversion of citrulline and aspartate to argininosuccinate which is catalysed by the argininosuccinate synthetase 1 (ASS1) gene. Argininosuccinate is then cleaved by argininosuccinate lyase 1 (ASL1) to produce arginine. In many types of tumours ASS1 is not expressed and consequently reduction in extracellular arginine can inhibit tumour growth, for example, 60-80% of melanomas do not express ASS1.

Degradation of arginine by arginine deiminase produces citrulline, which is the substrate for ASS1 to synthesize arginine, therefore cells such as T cells, which have an intact arginine biosynthetic pathway can use citrulline to produce arginine. Degradation of arginine with arginase produces ornithine which can also be used as an arginine precursor. Arginine decarboxylase produces agmatine from arginine modification, which modulates polyamine metabolism in cancer cells Thus modulating arginine may alter the microenvironment in favour of the immune response.

As mentioned above, cells such as T cells can use citrulline to produce arginine in a two-step enzymatic process involving the enzymes ASS and ASL (FIG. 4). Citrulline transport is mediated by the L-type amino acid transporter (LAT1). In the cells of the present invention, the expression of LAT1 may be upregulated. The cells of the present invention may comprise a heterologous nucleic acid sequence encoding LAT1.

A Mycoplasma derived enzyme, arginine deiminase, which catalyses the degradation of arginine into citrulline and ammonia. This enzyme has a very high affinity for arginine, but produces an immune reaction so is consequently pegylated to reduce immunogenicity. Human arginase has also been used in clinical trials, which degrades arginine to ornithine and urea. Citrulline is not metabolised by arginase.

A bacterial arginine biosynthesis pathway with enzymes from E. coli is shown in FIG. 5.

In one embodiment, the amino acid is arginine.

In one embodiment, the engineered cell (such as an engineered T-cell) secretes or expresses arginase, arginine deaminase and/or arginine decarboxylase.

Suitably, the engineered cell (such as an engineered T-cell) may secrete or express arginase. Suitably, the engineered cell (such as an engineered T-cell) may secrete or express arginine deaminase. Suitably, the engineered cell (such as an engineered T-cell) may secrete or express arginine decarboxylase.

In one embodiment, wherein the amino acid is arginine, the engineered cell (such as an engineered T-cell) secretes or expresses arginase, arginine deaminase and/or arginine decarboxylase.

An example of a human arginase is provided by UniProtKB Accession No: P05089;

(SEQ ID NO: 2; UniProtKB Accession No: P05089)
MSAKSRTIGIIGAPFSKGQPRGGVEEGPTVLRKAGLLEKLKEQECDVK
DYGDLPFADIPNDSPFQIVKNPRSVGKASEQLAGKVAEVKKNGRISLV
LGGDHSLAIGSISGHARVHPDLGVIWVDAHTDINTPLTTTSGNLHGQP
VSFLLKELKGKIPDVPGFSWVTPCISAKDIVYIGLRDVDPGEHYILKT
LGIKYFSMTEVDRLGIGKVMEETLSYLLGRKKRPIHLSFDVDGLDPSF
TPATGTPVVGGLTYREGLYITEEIYKTGLLSGLDIMEVNPSLGKTPEE
VTRTVNTAVAITLACFGLAREGNHKPIDYLNPPK.

An Example of an arginine deiminase from Mycoplasma arginine is provided by UniProtKB: P23793;

(SEQ ID NO: 3; UniProtKB Accession No: P23793)
MSVFDSKFKGIHVYSEIELESVLVHEPGREIDYITPARLDELLFSAI
LESHDARKEHKQFVAELKANDINVVELIDLVAETYDLASQEAKDKLI
EEFLEDSEPVLSEEHKVVVRNFLKAKKTSRELVEIMMAGITKYDLGI
EADHELIVDPMPNLYFTRDPFASVGNGVTIHYMRYKVRQRETLFSRF
VFSNHPKLINTPWYYDPSLKLSIEGGDVFIYNNDTLVVGVSERTDLQ
TVTLLAKNIVANKECEFKRIVAINVPKWTNLMHLDTWLTMLDKDKFL
YSPIANDVFKFWDYDLVNGGAEPQPVENGLPLEGLLQSIINKKPVLI
PIAGEGASQMEIERETHFDGTNYLAIRPGVVIGYSRNEKTNAALEAA
GIKVLPFHGNQLSLGMGNARCMSMPLSRKDVKW.

An example of a human arginine decarboxylase is provided by UniProtKB Accession No: B3KV62;

(SEQ ID NO: 4; UniProtKB Accession No: B3KV62)
MVLCIATDDSHSLSCLSLKFGVSLKSCRHLLENAKKHHVEVVGVSFH
IGSGCPDPQAYAQSIADARLVFEMGTELGHKMHVLDLGGGFPGTEGA
KVRFEEIASVINSALDLYFPEGCGVDIFAELGRYYVTSAFTVAVSII
AKKEVLLDQPGREEENGSTSKTIVYHLDEGVYGIFNSVLFDNICPTP
ILQKKPSTEQPLYSSSLWGPAVDGCDCVAEGLWLPQLHVGDWLVFDN
MGAYTVGMGSPFWGTQACHITYAMSRVAWEALRRQLMAAEQEDDVEG
VCKPLSCGWEITDTLCVGPVFTPASIM.

Example of a arginine decarboxylase from bacteria is provided by UniProtKB Accession No: Q57764 or Q9Z6M7.

An example of arginine decarboxylase from Arabidopsis thaliana is provided by UniProtKB Accession No: Q9S164;

(SEQ ID NO: 5; UniProtKB: Q9SI64)
MPALAFVDTPIDTFSSIFTPSSVSTAVVDGSCHWSPSLSSSLYRIDG
WGAPYFAANSSGNISVRPHGSNTLPHQDIDLMKVVKKVTDPSGLGLQ
LPLIVRFPDVLKNRLECLQSAFDYAIQSQGYDSHYQGVYPVKCNQDR
FIIEDIVEFGSGFRFGLEAGSKPEILLAMSCLCKGNPEAFLVCNGFK
DSEYISLALFGRKLELNTVIVLEQEEELDLVIDLSQKMNVRPVIGLR
AKLRTKHSGHFGSTSGEKGKFGLTTVQILRVVRKLSQVGMLDCLQLL
HFHIGSQIPSTALLSDGVAEAAQLYCELVRLGAHMKVIDIGGGLGID
YDGSKSGESDLSVAYSLEEYAAAVVASVRFVCDQKSVKHPVICSESG
RAIVSHHSVLIFEAVSAGQQHETPTDHQFMLEGYSEEVRGDYENLYG
AAMRGDRESCLLYVDQLKQRCVEGFKEGSLGIEQLAGVDGLCEWVIK
AIGASDPVLTYHVNLSVFTSIPDFWGIDQLFPIVPIHKLDQRPAARG
ILSDLTCDSDGKINKFIGGESSLPLHEMDNNGCSGGRYYLGMFLGGA
YEEALGGVHNLFGGPSVVRVLQSDGPHGFAVTRAVMGQSSADVLRAM
QHEPELMFQTLKHRAEEPRNNNNKACGDKGNDKLVVASCLAKSFNNM
PYLSMETSTNALTAAVNNLGVYYCDEAAAGGGGKGKDENWSYFG.

Phenylalanine

In some aspects, modifying amino acids to produce other bioactive molecules may have a dual effect; by reducing levels of amino acids but also producing alternate products which have biological functions relevant to tumour targeting.

For example, phenylalanine lyase degrades phenylalanine to cinnamic acid, which has been reported to possess anti-proliferative properties when added to cancer cells and depletion of phenylalanine has been shown to inhibit proliferation of murine leukaemic lymphoblasts.

Phenylalanine-ammonia lyase degrades phenylalanine to cinnamic acid. Cinnamic acid inhibits protiferation of tumour cells. Thus, modulating phenylalanine may alter the microenvironment in favour of the immune response.

In one embodiment, the amino acid is phenylalanine.

In one embodiment, the engineered cell (such as an engineered T-cell) secretes or expresses phenylalanine-ammonia lyase.

In one embodiment, wherein the amino acid is phenylalanine, the engineered cell (such as an engineered T-cell) secretes or expresses phenylalanine-ammonia lyase.

An example of a phenylalanine ammonia lyase from Arabidopsis thaliana (PAL1) is provided by UniProtKB Accession No: P35510;

(SEQ ID NO: 6; UniProtKB Accession No: P35510)
MEINGAHKSN GGGVDAMLCG GDIKTKNMVI NAEDPLNWGA AAEQMKGSHL
DEVKRMVAEF RKPVVNLGGE TLTIGQVAAI STIGNSVKVE LSETARAGVN
ASSDVVVMESM NKGTDSYGVT TGFGATSHRR TKNGVALQKE LIRFLNAGIF
GSTKETSHTL PHSATRAAML VRINTLLQGF SGIRFEILEA ITSFLNNNIT
PSLPLRGTIT ASGDLVPLSY IAGLLTGRPN SKATGPNGEA LTAEEAFKLA
GISSGFFDLQ PKEGLALVNG TAVGSGMASM VLFETNVLSV LAEILSAVFA
EVMSGKPEFT DHLTHRLKHH PGQIEAAAIM EHILDGSSYM KLAQKLHEMD
PLQKPKQDRY ALRTSPQWLG PQIEVIRYAT KSIEREINSV NDNPLIDVSR
NKAIHGGNFQ GTPIGVSMDN TRLAIAAIGK LMFAQFSELV NDFYNNGLPS
NLTASRNPSL DYGFKGAEIA MASYCSELQY LANPVTSHVQ SAEQHNQDVN
SLGLISSRKT SEAVDILKLM STTFLVAICQ AVDLRHLEEN LRQTVKNTVS
QVAKKVLTTG VNGELHPSRF CEKDLLKVVD REQVYTYADD PCSATYPLIQ
KLRQVIVDHA LINGESEKNA VTSIFHKIGA FEEELKAVLP KEVEAARAAY
DNGTSAIPNR IKECRSYPLY RFVREELGTE LLTGEKVTSP GEEFDKVFTA
ICEGKIIDPM MECLNEWNGA PIPIC.

Methionine

Several tumour types are dependent on methionine and many tumours have elevated S-adenosyl methionine requirements, therefore tumour cells require regeneration of methionine based intermediates. Defects in the methionine pathway have been reported in several tumour types, highlighting that tumour cells may be more dependent on external methionine. Although an essential amino acid, tumour cells can maintain levels by utilising salvage pathways or synthesizing methionine from homocysteine. For example, cells with PIKCA3 mutations have been shown to be sensitive to methionine depletion by downregulating the SLC7A11 gene which encodes a cysteine transporter. The result of this is to direct homocysteine towards cysteine synthesis thereby rendering cells sensitive to methionine depletion

In contrast, T-cells may be more resistant to low methionine levels as they may induce salvage pathways or may have lower methionine requirements when compared to tumour cells.

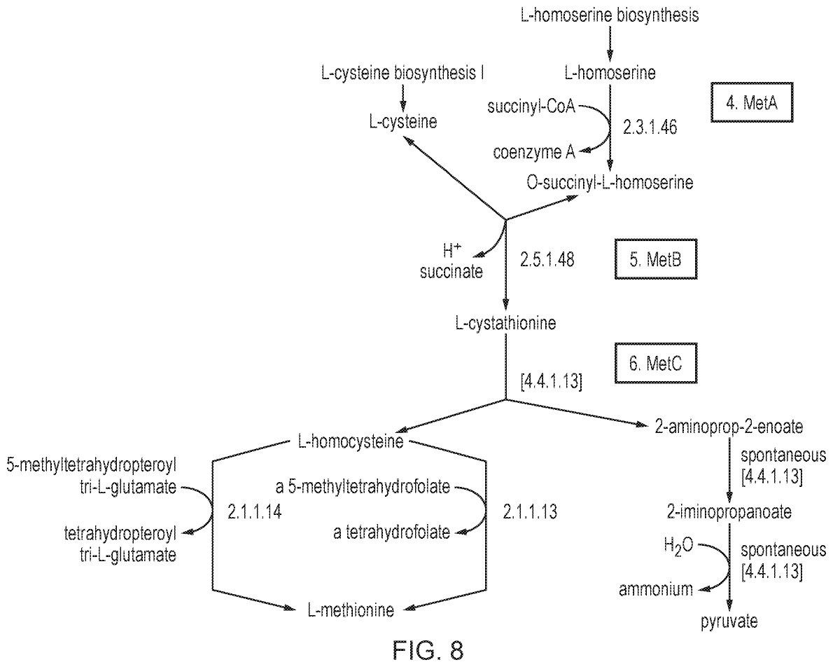

A bacterial methionine biosynthesis pathway with enzymes from E. coli is shown in FIG. 8. The present invention provides a T cell which is engineered to express thrA, asd, metA, metB, metC and/or metH. The T cell may be engineered to express metH or overexpress methionine synthase (MTR) to enhance the conversion of homocysteine to methionine by the T cells. Homocysteine may be given to the subject as a dietary supplement before or after T cell administration.

In one embodiment, the amino acid is methionine.

In one embodiment, the engineered cell (such as an engineered T-cell) secretes or expresses methioninase.

In one embodiment, wherein the amino acid is methionine, the engineered cell (such as an engineered T-cell) secretes or expresses methioninase.

An example of a L-methionine gamma-lyase from Pseudomonas putida is provided by UniProtKB Accession No: P13254:

(SEQ ID NO: 7; UniProtKB Accession No: P13254)
MHGSNKLPGFATRAIHHGYDPQDHGGALVPPVYQTATFTFPTVEYGAACFAGEQAGHFYSR
ISNPTLNLLEARMASLEGGEAGLALASGMGAITSTLWTLLRPGDEVLLGNTLYGCTFAFLH
HGIGEFGVKLRHVDMADLQALEAAMTPATRVIYFESPANPNMHMADIAGVAKIARKHGATV
VVDNTYCTPYLQRPLELGADLVVHSATKYLSGHGDITAGIVVGSQALVDRIRLQGLKDMTG
AVLSPHDAALLMRGIKTLNLRMDRHCANAQVLAEFLARQPQVELIHYPGLASFPQYTLARQ
QMSQPGGMIAFELKGGIGAGRRFMNALQLFSRAVSLGDAESLAQHPASMTHSSYTPEERAH
YGISEGLVRLSVGLEDIDDLLADVQQALKASA.

Threonine

Degradation of threonine by threonine deaminase to ammonia and ketobutyrate has been shown to be cytotoxic towards leukemic cells and appeared to be more efficient than removing threonine from culture medium (Greenfield and Wellner, 1977).

Threonine can also be depleted by threonine dehydrogenase which converts threonine to ketobutyrate and NADH, either enzyme can be used to deplete threonine levels in the culture medium, which can be monitored by ELISA assay.

An example of a threonine deaminase is provided by UniProtKB Accession No: P20132;

(SEQ 10 NO: 8; UniProtKB Accession No: P20132)
MMSGEPLHVKTPIRDSMALSKMAGTSVYLKMDSAQPSGSFKIRGIGHFCKRWAKQGCAHFV
CSSAGNAGMAAAYAARQLGVPATIVVPSTTPALTIERLKNEGATVKVVGELLDEAFELAKA
LAKNNPGWVYIPPFDDPLIWEGHASIVKELKETLWEKFGAIALSVGGGGLLCGVVQGLQEV
GWGDVPVIAMETFGAHSFHAATTAGKLVSLFKITSVAKALGVKTVGAQALKLFQEHPIFSE
VISDQEAVAAIEKFVDDEKILVERACGAALAAVYSHVIQKLQLEGNLRTPLPSLVVIVCGG
SNISLAQLRALKEQLGMTNRLPK.

An example of a threonine dehydrogenase (Mus musculus) is provided by UniProtKB Accession No: Q8K3F7;

(SEQ ID NO: 9; UniProtKB Accession No: Q8K3F7)
MLFLGMLKQVVNIGTAQSKASSCRKLVLPLKFLGTSQHRIPADANFHSTSISEAEPPRV
LITGGLGQLGVGLANLLRKRFGKDNVILSDIRKPPAHVFHSGPFVYANILDYKSLREIW
NHRISWLFHYSALLSAVGEANVSLARDVNITGLHNIVLDVAAEYNVRLFVPSTIGAFGP
TSPRNPAPDLCIQRPRTIYGVSKVHTELMGEYYYYRYGLDFRCLRYPGIISADSQPGGG
ITDYAVCIIFHAAAKNGTFECNLEAGTRLPMMYISDCLRATLEVMEAPAERLSMRTYNI
SAMSFTPEELAQALRKHAPDFQITYCVDPLRQAIAESWPMILDDSNARKDWGWKHDFDL
PELVATMLNFHGVSTRVAQVN.

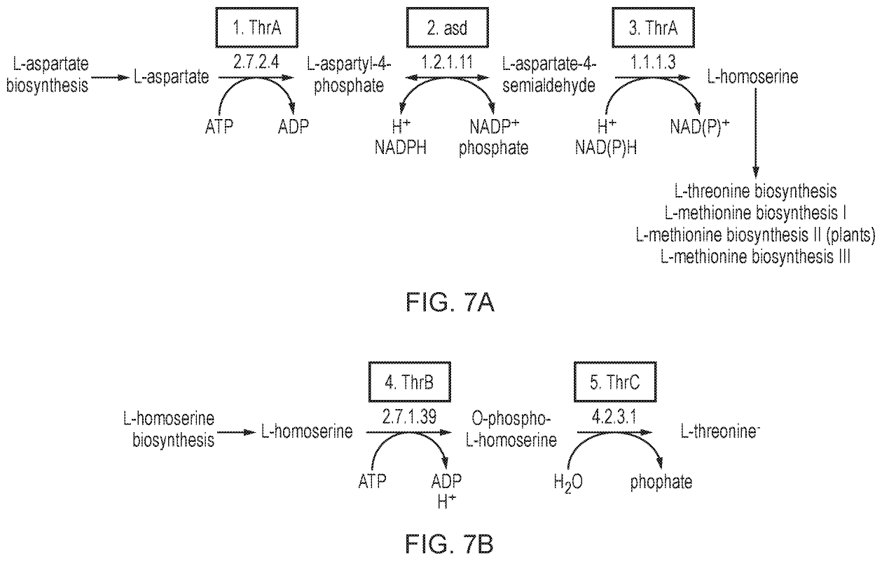

A bacterial threonine biosynthesis pathway with enzymes from E. coli is shown in FIG. 7. The present invention provides a T cell which is engineered to express thrA, asd, thrB and/or thrC.

Leucine

Leucine depletion has been reported to inhibit the growth of breast cancer (MD-MD 231) and Melanoma (A2058, SK-MEL3) cell lines, particularly those driven by Ras-MEK pathway mutations.

Leucine can be degraded by branched chain amino-acid aminotransferase (human cytoplasmic form) or leucine dehydrogenase (bacterial).

An example of a human branched chain amino acid aminotransferase BCAT1 is provided by UniProtKB Accession No: P54687;

(SEQ ID NO: 10; UniProtKB Accession No: P54687)
MKDOSNGCSAECTGEGGSKEVVGTFKAKDLIVTPATILKEKPDPNNLVFGTVFIDHMLI
VEWSSEFGWEKPHIKPLQNLSLHPGSSALHYAVELFEGLKAFRGVDNKIRLFQPNLNIM
DRMYRSAVRATLPVFDKEELLECIQQLVKLDQEMPYSTSASLYIRPTFIGTEPSLGVKK
PTKALLFVLLSPVGPYFSSGTFNPVSLWANPKYVRAWKGGIGDCKMGGNYGSSLFAQCE
AVDNGCQQVLWLYGEDHCIITEVGTMNLFLYWINEDGEEELATPPLDGIILPGVIRRCI
LDLAHCIWGEFKVSERYLTMDDLTTALEGNRVREMFGSGTACWCPVSDILYKGETIHIP
TMENGPKLASRILSKLTDIQYGREESDWTIVLS.

An example of a leucine dehydrogenase from Thermoactinomyces intermediusis provided by UniProtKB Accession No: Q60030

(SEQ ID NO: 11; UniProtKB Accession No: 060030)
MKIFDYMEKYDYEQLVMCQDKESGLKAIICIHVTTLGPALGGMRMWIYASEEEAIEDAL
RLGRGMTYKNAAAGLNLGGGKMIGDPRKDKNEAMFRALGRFIQGLNGRYITAEDVGITV
EDMDIIFIEETRYVTGVSPAFGSSGNPSPVTAYGVYRGNIKAAAKEAFGDDSLEGKWAV
QGVGHVAYELCKHLHNEGAKLIVTDINKENADRAVQEFGAEFVHPDKIYDVECDIFAPC
ALGAIINDETIEFLKCKWAGSANNQLKEERHGKMLEEKGIWAPDYVINAGGVINVADEL
LGYNRERAMKKVEGIYDKILKVFEIAKRDGIPSYLAADRMAEERIEMMRKTRSTFLQDQ
RNLINFNNK.

The Adenosine Pathway

In another embodiment, the molecule is a nucleotide or nucleoside. Suitably, the molecule may be a nucleotide. Suitably, the molecule may be a nucleoside.

Nucleotides (such as ATP and AMP) are broken down to adenosine via ecto-nucleotidase reactions (such as via CD39 and CD73 respectively). Adenosine levels are thought to be raised in numerous cancer tissues. Adenosine is immunosuppressive and modification of the adenosine metabolic pathway creates an immune tolerant microenvironment which promotes tumour growth and progression.

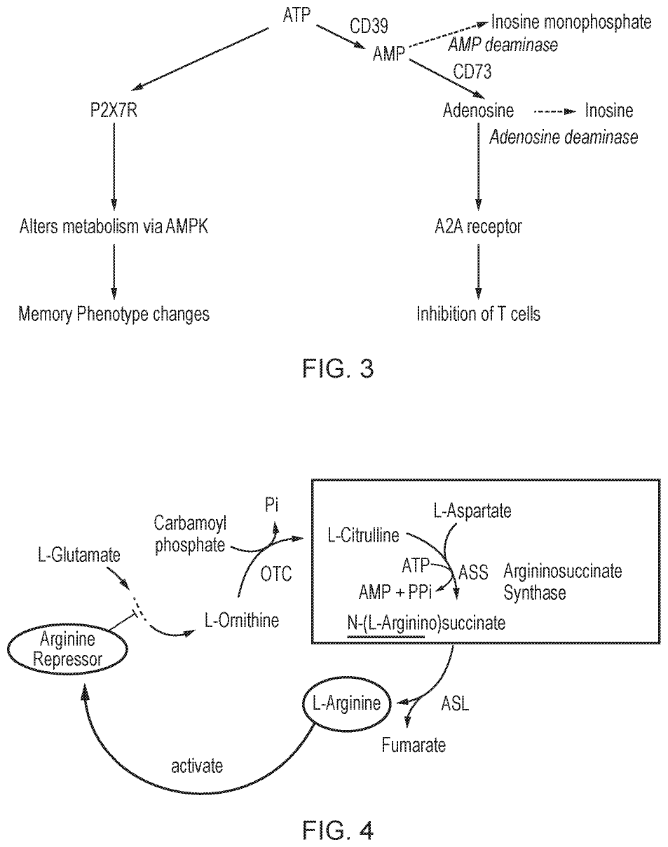

Adenosine signalling is also thought to affect chemoresistance in some tumours, the expression of ASS1 has been linked to cisplatin sensitivity. A schematic diagram of the adenosine pathway is shown in FIG. 3.

In the context of the present invention, the molecule may be an adenosine metabolite.

In one embodiment, the molecule is adenosine.

In one embodiment, the engineered cell (such as an engineered T-cell) secretes or expresses adenosine deaminase or AMP deaminase.

In one embodiment, wherein the molecule is adenosine, the engineered cell (such as an engineered T-cell) secretes or expresses adenosine deaminase or AMP deaminase.

An example of a human Amp deaminase is provided by UniProtKB Accession No: Q01432;

(SEQ 10 NO: 12; UniProtKB Accession No: Q01432)
MPRQFPKLNISEVDEQVRLLAEKVFAKVLREEDSKDALSLFTVPEDCPIGQKEAKEREL
QKELAEQKSVETAKRKKSFKMIRSQSLSLQMPPQQDWKGPPAASPAMSPTTPVVTGATS
LPTPAPYAMPEFQRVTISGDYCAGITLEDYEQAAKSLAKALMIREKYARLAYHRFPRIT
SQYLGHPRADTAPPEEGLPDFHPPPLPQEDPYCLDDAPPNLDYLVHMQGGILFVYDNKK
MLEHQEPHSLPYPDLETYTVDMSHILALITDGPTKTYCHRRLNFLESKFSLHEMLNEMS
EFKELKSNPHRDFYNVRKVDTHIHAAACMNQKHLLRFIKHTYQTEPDRTVAEKRGRKIT
LRQVFDGLHMDPYDLTVDSLDVHAGRQTFHRFDKFNSKYNPVGASELRDLYLKTENYLG
GEYFARMVKEVARELEESKYQYSEPRLSIYGRSPEEWPNLAYWRIQHKVYSPNMRWIIQ
VPRIYDIFRSKKLLPNFGKMLENIFLPLFKATINPQDHRELHLFLKYVTGFDSVDDESK
HSDHMFSDKSPNPDVWTSEQNPPYSYYLYYMYANIMVLNNLRRERGLSTFLFRPHCGEA
GSITHLVSAFLTADNISHGLLLKKSPVLQYLYYLAQIPIAMSPLSNNSLFLEYSKNPLR
EFLHKGLHVSLSTDDPMQFHYTKEALMEEYAIAAQVWKLSTODLCEIARNSVLQSGLSH
QEKQKFLGQNYYKEGPEGNDIRKTNVAQIRMAFRYETLCNELSFLSDAMKSEEITALTN.

An example of a human adenosine deaminase is provided by UnlProtKB Accession No: P00813;

(SEQ ID NO: 13: UniProtKB Accession No: P00813)
MAQTPAFDKPKVELHVFILDGSIKPETILYYGRRRGIALPANTAEGLLNVIGNIDKPLU
PDFLAKFDYYMPAIAGCREAIKRIAYEFVEMKAKEGVVYVEVRYSPFILLANSKVEPIP
MQAEGDLTPDEVVALVGQGLQEGERDFGVKARSILCCMRHQPNWSPKVVELCKKYQQQT
VVAIDLAGDETIPGSSLLPGHVQAYQEMIKSGIHRTVHAGEVGSAEINKEAVDILKTER
LLGHGYHTLEDQALYNRRQENMHFEICPWSSYLTGAWKPDTEHAVIRLKNDQANYSLNT
DDPLIFKSTLDTDYQUITKRDMGFTEEEFKRLNINAAKSSFLPEDEKRELLDLLYKAYG
MPPSASAGQNL.

The Kynurenine Pathway

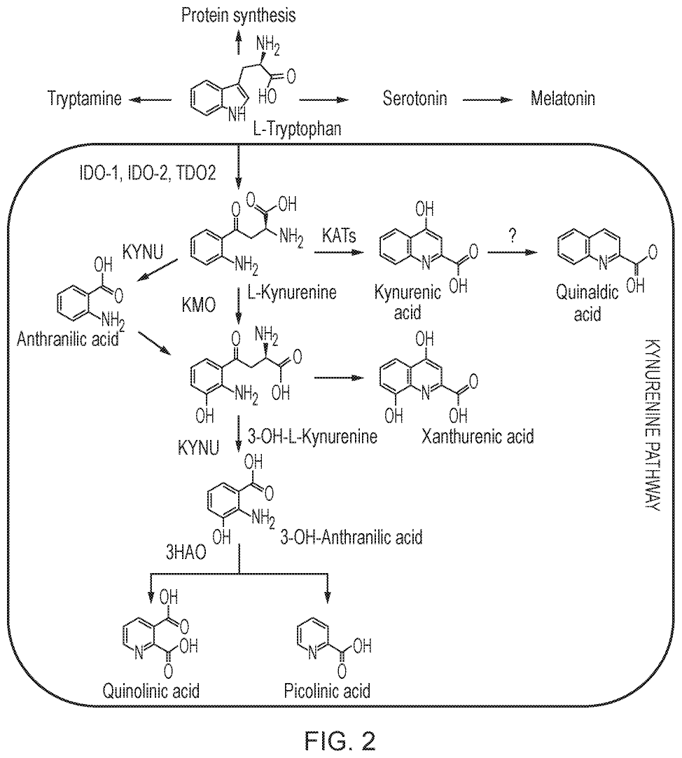

The tumour microenvironment sustains a strong immunosuppressive activity, maintained in part by production tryptophan metabolites within the microenvironment. The pathway of degradation of tryptophan to produce immunosuppressive products is shown in FIG. 2. One of these metabolites, kynurenine acts by binding to the AHR and stimulating transcription via XRE sequences.

In the context of the present invention, the molecule may be a tryptophan metabolite. In one embodiment, the tryptophan metabolite is kynurenine.

In one embodiment, the engineered cell (such as an engineered T-cell) secretes or expresses kynureninase.

An example of a kynureninase is provided by UniProtKB Accession No: Q16719;

(SEQ ID NO: 14; UniProtKB Accession No: Q16719)
MEPSSLELPADTVQRIAAELKCHPTDERVALHLDEEDKLRHFRECFYIPKIQDLPPVDL
SLVNKDENAIYFLGNSLGLQPKMVKTYLEEELDKWAKIAAYGHEVGKRPWITGDESIVG
LMKDIVGANEKEIALMNALTVNLHLLMLSFFKPTPKRYKILLEAKAFPSDHYAIESQLQ
LHGLNIEESMRMIKPREGEETLRIEDILEVIEKEGDSIAVILFSGVHFYTGQHFNIPAI
TKAGQAKGCYVGFDLAHAVGNVELYLHDWGVDFACWCSYKYLNAGAGGIAGAFIHEKHA
HTIKPALVGWFGHELSTRFKMDNKLQUPGVCGFRISNPPILLVCSLHASLEIFKQATMK
ALRKKSVLLTGYLEYLIKHNYGKDKAATKKPVVNIITPSHVEERGCQLTITFSVPNKDV
FQELEKRGVVCDKRNPNGIRVAPVPLYNSFHDVYKFTNLLTSILDSAETKN.

An example of an enzyme which modifies amino acids, including tryptophan is aromatic acid decarboxylase, which acts on amino acids possessing an aromatic side chain. This enzyme modifies tryptophan to tryptamine which has been shown to inhibit indole dioxygenase (IDO) an enzyme which produces kynurenine metabolites which are inhibitory to T cell function and phenylalanine to phenyl ethylamine, which has been reported to have some effect on lymphocyte function.

An example of a human aromatic acid decarboxylase is provided by UniProtKB Accession No: P20711;

(SEQ ID NO: 15; UniProtIKB Accession No: P20711)
MNASEFRRRGKEMVDYMANYMEGIEGRQVYPDVEPGYLRPLIPAAAPQEPDTFEDIIND
VEKIIMPGVTHVVHSPYFFAYFPTASSYPAMLADMLCGAIGCIGFSWAASPACTELETV
MMDWLGKMLELPKAFLNEKAGEGGGVIQGSASEATLVALLAARTKVIHRLQAASPELTQ
AAIMEKLVAYSSDQAHSSVERAGLIGGVKLKAIPSDGNFAMRASALQEALERDKAAGLI
PFFMVATLGTTTCCSFDNLLEVGPICNKEDIWLHVDAAYAGSAFICPEFRHLLNGVEFA
DSFNFNPHKWLLVNFDCSAMWVKKRTDLTGAFRLDPTYLKHSHQDSGLITDYRHWQIPL
GRRFRSLKMWFVFRMYGVKGLQAYIRKHVQLSHEFESLVRQDPRFEICVEVILGLVCFR
LKGSNKVNEALLQRINSAKKIHLVPCHLRDKFVLRFAICSRTVESAHVQRAWEHIKELA
ADVLRAERE.

Lipids

Cancer cells exhibit increased demand for fatty acids and increased rates of lipid synthesis occur through increased expression of various lipogenic enzymes. Increased lipid production appears to be critical for cancer cell survival. In some tumours, such as prostate tumours, beta-oxidation of fatty acids is thought to be an important alternative energy source to glucose. Sphingosine-1-phosphate (S1P) has been shown to affect proliferation in ovarian cell lines (SKOV3). Lysophosphatidic acid (LPA) is a potent mitogen which has been shown to affect tumour cell proliferation. It has been reported that LPA also inhibits T cell activation, therefore, decreasing levels of LPA in the tumour microenvironment would have a twofold benefit, inhibiting tumour growth and stimulating T cell activation. Thus modulating lipids in the microenvironment may promote the immune response.

In one embodiment, the molecule is a lipid.

Suitably, the lipid may be selected from the following group: Prostaglandin E2 (PGE2), Sphingosine-1-phosphate (S-1-P) and Lysophosphatidic acid (LPA). Suitably, the lipid may be Prostaglandin E2 (PGE2). Suitably, the lipid may be Sphingosine-1-phosphate (S-1-P). Suitably, the lipid may be Lysophosphatidic acid (LPA).

Suitably, PGE2 may be degraded by 15-hydroxyprostaglandin dehydrogenase (15-PGDH).

An example of 15-PGDH is provided by UniProtKB Accession No P15428;

(SEQ ID NO: 16; UniProtKB Accession No: P15428)
MHVNGKVALVTGAAQGIGRAFAEALLLKGAKVALVDWNLEAGVQCKAALDEQFEPQKTLFIQ
CDVADQQQLRDTFRKVVDHFGRLDILVNNAGVNNEKNWEKTLQINLVSVISGTYLGLDYMSK
QNGGEGGIIINMSSLAGLMPVAQQPVYCASKHGIVGFTRSAALAANLMNSGVRLNAICPGFV
NTAILESIEKEENMGQYIEYKDHIKDMIKYYGILDPPLIANGLITLIEDDALNGAIMKITTS
KGIHFQDYDTTPFQAKTQ.

Suitably, S-1-P may be degraded by S-1-P lyase.

An example of a human S-1-P lyase is provided by UniProtKB Accession No: O95470:

(SEQ ID NO: 17; UniProtKB Accession No: 095470)
MPSTDLLMLKAFEPYLEILEVYSTKAKNYVNGHCTKYEPWQLIAWSVVVVTLLIVWGYE
FVFQPESLWSRFKKKCFKLTRKMPIIGRKIQDKLNKTKDDISKNMSFLKVDKEYVKALP
SQGLSSSAVLEKLKEYSSMDAFWQEGRASGTVYSGEEKLTELLVKAYGDFAWSNPLHPD
IFPGLRKIEAEIVRIACSLFNGGPDSCGCVTSGGTESILMACKAYRDLAFEKGIKTPEI
VAPQSAHAAFNKAASYFGMKIVRVPLTKMMEVDVRAMRRAISRNTAMLVCSTPQFPHGV
IDPVPEVAKLAVKYKIPLHVDACLGGFLIVFMEKAGYPLEHPFDFRVKGVTSISADTHK
YGYAPKGSSLVLYSDKKYRNYQFFVDTDWQGGIYASPTIAGSRPGGISAACWAALMHFG
ENGYVEATKQIIKTARFLKSELENIKGIFVFGNPQLSVIALGSRDFDIYRLSNLMTAKG
WNLNQLQFPPSIHFCITLLHARKRVAIQFLKDIRESVTQIMKNPKAKTTGMGAIYGMAQ
TTVDRNMVAELSSVFLDSLYSTDTVTQGSQMNGSPKPH

Suitably, LPA may be degraded by lipid phosphate phosphatases.

An example of a human phospholipid phosphatase is provided by UniProtKB Accession No: O14494:

(SEQ ID NO:18; UniProtKB Accession No: 014491)
MFDKTRLPYVALDVLCVLLAGLPFAILTSRHTPFQRGVFCNDESIKYPYKEDTIPYALL
GGIIIPFSIMILGETLSVYCNLLHSNSFIRNNYIATIYKAIGTFLFGAAASQSLTDIAK
YSIGRLRPHFLDVCDPDWSKINCSDGYIEYYICRGNAERVKEGRLSFYSGHSSFSMYCM
LFVALYLQARMKGDWARLLRPTLQFGLVAVSIYVGLSRVSDYKHHWSDVLTGLIQGALV
AILVAVYVSDFFKERTSFKERKEEDSHTTLHETPTTGNHYPSNHQP.

An example of a human phospholipid phosphatase 3 is provided by UniProtKB Accession No: O14495;

(SEQ ID NO: 19; UniProtKB Accession No: 014495)
MQNYKYOKAMDESKNGGSPALNNNPRRSGSKRVLLICLDLFCLFMAGLPFLIIETSTIKPYHR
GFYCNDESIKYPLKTGETINDAVLCAVGMAILAIITGEFYRIYYLKKSRSTIQNPYVAALYKQVG
CFLFGCAISQSFIDIAKVSIGRLRPHFLSVONPDFSQINCSEGYIQNYRCRGDDSKVQEARKS
FFSGHASFSMYTMLYLVLYWARFTWRGARLIRPLIQFTLIMMAFYTGLSRVSDHKFAHPSDV
LAGFAQGALVACCIVFFVSDLFKTKTTLSLPAPAIRKEILSPVDIORNNFAHNMM.

Product

In one embodiment, said enzyme(s) converts the molecule into a product which is selected from an amino acid or derivative thereof, a nucleotide or nucleoside or derivatives thereof or a lipid or derivative thereof.

In one aspect, said enzyme(s) converts the molecule into a product which is detrimental to the survival or proliferation of a tumour cell or promotes the proliferation and/or activity of the engineered cell (such as engineered T-cell).

As used herein “product which is detrimental to” means that in the presence of the product, the survival or proliferation of the tumour cell (or population of tumour cells) is compromised, reduced or completely abolished.

Suitably, in the presence of the product, the survival and or proliferation of the tumour cell (or population of tumour cells) may be reduced by at least 5%, at least 10%, at least 20%, at least 30%, at least 40%, at least 50%, at least 60%, at least 70%, at least 80%, at least 90%, at least 95% or at least 99%.

As used herein, “promotes the proliferation and/or activity of the engineered cell” means that the proliferation or activity of the engineered cell (or population of engineered cells) is unchanged or increased.

Suitably, in the presence of the product, the proliferation and/or activity of the engineered cell (or population of engineered cells) may be increased by at least 5%, at least 10%, at least 20%, at least 30%, at least 40%, at least 50%, at least 60%, at least 70%, at least 80%, at least 90%, at least 95% or at least 99% compared with the proliferation/activity in the absence of the product.

In one aspect, the product may be selected from: agmatine, tryptamine, dimethyltryptamine, tyramine, histamine, phenylethylamine or cinnamic acid. Suitably, the product may be agmatine. Suitably, the product may be tryptamine. Suitably, the product may be dimethyltryptamine. Suitably, the product may be tyramine. Suitably, the product may be histamine. Suitably, the product may bephenylethylamine. Suitably, the product may be cinnamic acid.

Cells may be engineered to survive in the absence of a molecule in the extracellular environment, thus providing them with a survival advantage in the absence of said molecule. This concept may be used to selectively kill tumour cells whilst retaining engineered cells (such as engineered T-cells) in the microenvironment. Thus, engineering a cell to survive in the absence of a molecule in the extracellular environment may enable the microenvironment to be altered in favour of the immune response.

In one aspect, the engineered cell (such as an engineered T-cell) is engineered to survive in the absence of the molecule in the extracellular environment.

Suitably, the cell may be engineered to:

synthesise the molecule or a precursor thereof intracellularly;
inhibit the intracellular breakdown of the targeted products; and/or
increase the efficiency of the import of the molecule or precursor thereof.

It will be understood that any combination of the above-mentioned methods may render the engineered cell resistant to the absence of the molecule in the extracellular environment.

In one embodiment, the cell may be engineered to synthesise tryptophan. Suitably the cell may be engineered to synthesise tryptophan intracellularly.

Suitably, the cell may be engineered to synthesize tryptophan intracellularly, wherein the molecule is a tryptophan metabolite (such as kynurenine) and/or the cell secretes kynureninase or expresses kynureninase at its cell surface.

Cell

An “engineered cell” as used herein means a cell which has been modified to comprise or express a nucleic acid sequence which is not naturally encoded by the cell. Methods for engineering cells are known in the art and include but are not limited to genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell.

Accordingly, the nucleic acid sequence encoding the CAR, TCR or enzyme is not naturally expressed by a corresponding, unmodified cell.

Suitably, an engineered cell is a cell whose genome has been modified e.g. by transduction or by transfection. Suitably, an engineered cell is a cell whose genome has been modified by retroviral transduction. Suitably, an engineered cell is a cell whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector.

Engineered cells according to the present invention may be generated by introducing DNA or RNA coding a CAR or engineered TCR and one or more enzymes which when secreted or expressed at the cell surface, causes depletion of a molecule extracellular to the engineered cell; wherein said molecule is selected from: an amino acid; a nucleotide or nucleoside; or a lipid;

by one of many means including transduction with a viral vector, transfection with DNA or RNA.

Cells may be activated and/or expanded prior to the introduction of a nucleic acid sequence, for example by treatment with an anti-CD3 monoclonal antibody or both anti-CD3 and anti-CD28 monoclonal antibodies. As used herein “activated” means that a cell has been stimulated, causing the cell to proliferate, differentiate or initiate an effector function.

Methods for measuring cell activation are known in the art and include, for example, measuring the expression of activation markers by flow cytometry, such as the expression of CD69, CD25, CD38 or HLA-DR or measuring intracellular cytokines.

As used herein “expanded” means that a cell or population of cells has been induced to proliferate.

The expansion of a population of cells may be measured for example by counting the number of cells present in a population. The phenotype of the cells may be determined by methods known in the art such as flow cytometry.

In one embodiment, the engineered cell according to the present invention may be an engineered immune effector cell.

An “immune effector cell” as used herein is a cell of the immune system which responds to a stimulus and effects a change.

Suitably, an immune effector cell may a T-cell (such as an alpha-beta T-cell or a gamma-delta T-cell), a B cell (such as a plasma cell), a Natural Killer (NK) cell or a macrophage.

In one embodiment, the engineered cell according to the present invention may be an engineered cytolytic immune cell.

“Cytolytic immune cell” as used herein is a cell which directly kills other cells. Cytolytic cells may kill cancerous cells; virally infected cells or other damaged cells. Cytolytic immune cells include T-cells and Natural killer (NK) cells.

Cytolytic immune cells can be T-cells or T lymphocytes which are a type of lymphocyte that play a central role in cell-mediated immunity. T-cells can be distinguished from other lymphocytes, such as B cells and NK cells, by the presence of a TCR on their cell surface.

Cytolytic T-cells (TC cells, or CTLs) destroy virally infected cells and tumour cells, and are also implicated in transplant rejection. CTLs express the CD8 at their surface. CTLs may be known as CD8+ T-cells. These cells recognize their targets by binding to antigen associated with MHC class I, which is present on the surface of all nucleated cells. Through IL-10, adenosine and other molecules secreted by regulatory T-cells, the CD8+ cells can be inactivated to an anergic state, which prevent autoimmune diseases such as experimental autoimmune encephalomyelitis.

Suitably, the engineered cell of the present invention may be a T-cell. Suitably, the T-cell may be an alpha-beta T-cell. Suitably, the T-cell may be a gamma-delta T-cell.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

Suitably, the engineered cell of the present invention may be a wild-type killer (NK) cell. Suitably, the cell of the present invention may be a cytokine induced killer cell.

The engineered cell according to the present invention may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the engineered cell according to the present invention may be derived from ex vivo differentiation of inducible progenitor cells or embryonic progenitor cells to T-cells. Alternatively, an immortalized T-cell line which retains its lytic function may be used.

Chimeric Antigen Receptor

The present invention provides an engineered cell (such as an engineered T-cell) which expresses a chimeric antigen receptor (CAR) together with one or more enzymes.

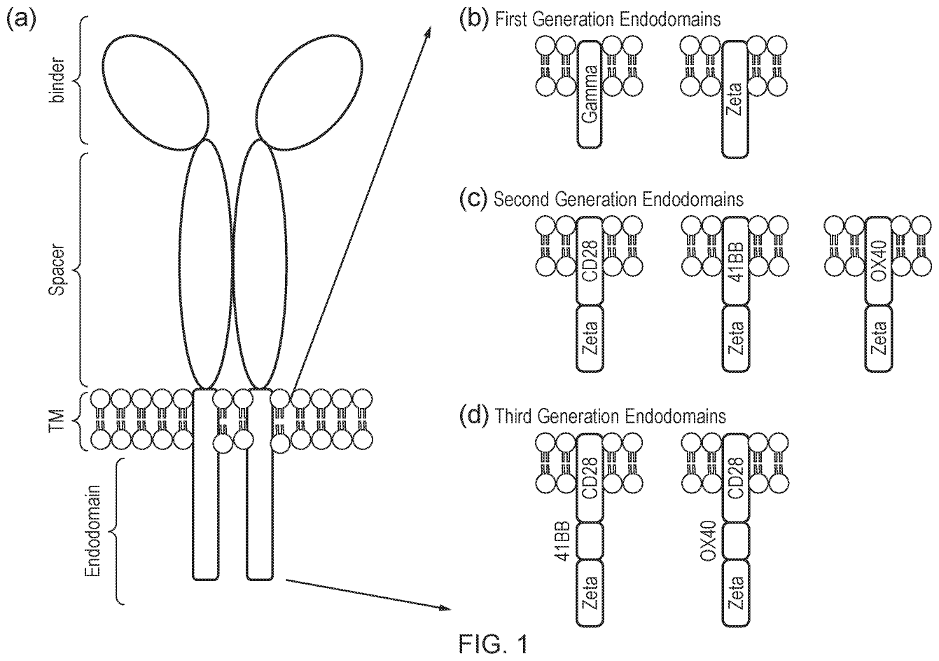

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3 results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T-cells using, for example, retroviral vectors. In this way, a large number of antigen-specific T-cells can be generated for adoptive cell transfer. When the CAR binds the target-antigen, this results in the transmission of an activating signal to the T-cell it is expressed on. Thus the CAR directs the specificity and cytotoxicity of the T-cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table. The antigen-binding domain used in the present invention may be a domain which is capable of binding a TAA as indicated therein.

TABLE 2

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The antigen-binding domain may comprise a proliferation-inducing ligand (APRIL) which binds to B-cell membrane antigen (BCMA) and transmembrane activator and calcium modulator and cyclophilin ligand interactor (TACI). A CAR comprising an APRIL-based antigen-binding domain is described in WO2015/052538.

Transmembrane Domain

The transmembrane domain is the sequence of a classical CAR that spans the membrane. It may comprise a hydrophobic alpha helix. The transmembrane domain may be derived from CD28, which gives good receptor stability.

CAR or TCR Signal Peptide

The CAR or engineered TCR for use in to the present invention may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The receptor may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T-cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The intracellular signalling domain may be or comprise a T-cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix(6-8)YxxL/I).

ITAMs are important for signal transduction in immune cells. Hence, they are found in the tails of important—cell signalling molecules such as the CD3 and ζ-chains of the T-cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The intracellular signalling domain component may comprise, consist essentially of, or consist of the CD3-ζ endodomain, which contains three ITAMs. Classically, the CD3-ζ endodomain transmits an activation signal to the T-cell after antigen is bound. The intracellular signalling domain may comprise additional co-stimulatory signalling. For example, 4-1BB (also known as CD137) can be used with CD3-ζ, or CD28 and OX40 can be used with CD3-ζ to transmit a proliferative/survival signal.

Transgenic T-Cell Receptor (TCR)

The present invention provides an engineered cell which expresses an engineered T-cell receptor (TCR) and one or more enzymes which, when secreted or expressed at the cell surface, causes depletion of a molecule extracellular to the engineered cell wherein said molecule is selected from: an amino acid; a nucleotide or nucleoside; or a lipid.

The T-cell receptor (TCR) is a molecule found on the surface of T-cells which is responsible for recognizing fragments of antigen as peptides bound to major histocompatibility complex (MHC) molecules.

The TCR is a heterodimer composed of two different protein chains. In humans, in 95% of T-cells the TCR consists of an alpha (α) chain and a beta (β) chain (encoded by TRA and TRB, respectively), whereas in 5% of T-cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T-cells and transferred back into patients for T-cell adoptive therapies. Such ‘heterologous’ TCRs may also be referred to herein as ‘transgenic TCRs’.

The transgenic TCR for use in the present invention may recognise a tumour associated antigen (TAA) when fragments of the antigen are complexed with major histocompatibility complex (MHC) molecules on the surface of another cell.

Suitably, the transgenic TCR for use in the present invention may recognise a TAA listed in Table 2.

Nucleic Acid Construct/Kit of Nucleic Acid Sequences

The present invention also provides a kit of polynucleotides comprising: (i) a first polynucleotide which encodes an enzyme which, when secreted or expressed at the cell surface, causes depletion of a molecule extracellular to the cell; and (ii) a second polynucleotide which encodes a chimeric antigen receptor (CAR) or a transgenic T-cell receptor (TCR).

The present invention provides a kit of polynucleotides comprising: (i) a first polynucleotide which encodes one or more enzymes involved in the intracellular synthesis of a molecule; and (ii) a second polynucleotide which encodes a chimeric antigen receptor (CAR) or a transgenic T-cell receptor (TCR).

The molecule may be required by a tumour cell for survival, proliferation, metastasis or chemoresistance. The molecule may be an amino acid; a nucleotide or nucleoside; or a lipid, or a derivative thereof. The molecule may be an amino acid such as an essential amino acid. The molecule may be isoleucine, leucine, lysine, methionine, arginine, phenylalanine, threonine, tryptophan or valine.

The one or more enzyme(s) may be a bacterial enzyme, such as one of the enzymes involved in the biosynthetic pathways shown in FIGS. 5 to 10.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The kit may comprise one nucleic acid sequence under the control of a constitutively active promoter and one nucleic acid sequence under the control of a selectively active promoter.

The kit may comprise two nucleic acid sequences under the control of different selectively active promoters.

The kit may comprise two nucleic acid sequences, one which comprises a specific miRNA target sequence and one which doesn't.

The kit may comprise two nucleic acid sequences comprising different miRNA target sequences.

One or both nucleic acid sequences may comprise a combination of a selectively active promoter and an miRNA target sequence.

The present invention also provides a cassette or nucleic acid construct comprising two or more nucleic acid sequences, a nucleic acid construct which comprises: (i) a first polynucleotide which encodes an enzyme as defined herein; and (ii) a second polynucleotide which encodes a chimeric antigen receptor (CAR) or a transgenic T-cell receptor (TCR).

Suitably, the cassette or nucleic acid construct may comprise a plurality of nucleic acid sequences which encode one or more enzymes as defined herein; and a CAR or transgenic TCR. For example, the nucleic acid construct may comprise two, three, four or more nucleic acid sequences which encode different components of the invention.

The plurality of nucleic acid sequences may be separated by co-expression sites.

The nucleic acid construct may comprise one nucleic acid sequence under the control of a constitutively active promoter and one nucleic acid sequence under the control of a selectively active promoter.

The nucleic acid construct may comprise two nucleic acid sequences under the control of different selectively active promoters.

Expression cassettes can be engineered to incorporate split transcriptional systems. For example, the vector can express two separate transcripts. A 5′ selectively active promoter may drive transcription of a long transcript where the first open reading frame codes for a first protein which is selectively expressed. Downstream from this, a second constitutively active promoter in the same orientation as the first may drive transcription of a shorter transcript where a second open reading frame codes for a second protein which is constitutively expressed. Both transcripts share the same polyA adenylation signal.

Alternatively, two separate promoters can drive expression of two independent transcripts. The transcripts may be oriented head-to-head in which one transcript reads from the sense strand and the other reads from the anti-sense strand. Alternatively, a constitutively active bi-directional promoter may be used which results in transcription of two transcripts in opposite direction. Each transcript may be controlled separately.

Cells can be engineered with combination of cassettes which have independent expression controlled either by promotors or miRNA target sequences, or both.

More conveniently, cells can be engineered with single cassettes which allow differential expression of different transgenes. For instance, a retroviral vector cassette can transcribe two transcripts one which is constitutively expressed and one which is conditionally expressed.

Co-Expression Site

A co-expression site is used herein to refer to a nucleic acid sequence enabling co-expression of nucleic acid sequences encoding the one or more enzymes described herein and a CAR or transgenic TCR according to the present invention.

Suitably, there may be a co-expression site between the nucleic acid sequence encoding the one or more enzymes and the nucleic acid sequence which encodes the CAR or transgenic TCR. Suitably, in embodiments where a plurality of co-expression sites is present in the engineered polynucleotide, the same co-expression site may be used.

Preferably, the co-expression site is a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site. Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide. A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expression sequence may be an internal ribosome entry sequence (IRES). The co-expressing sequence may be an internal promoter.

Promoters

The term “promoter” used herein means a promoter and/or enhancer. A promoter is a region of DNA that initiates transcription of a particular gene. Promoters are located near the transcription start sites of genes, on the same strand and upstream on the DNA (towards the 5′ region of the sense strand). Promoters are usually about 100-1000 base pairs long. An enhancer is a short (50-1500 bp) region of DNA that can be bound by transcription factors to increase the likelihood that transcription of a particular gene will occur. Enhancers are cis-acting and can be located upstream or downstream from the transcription start site.

Using Selective Expression to Optimise Cell Function

The nucleic acid sequence(s) or construct(s) of the invention may be designed to optimise cell function. Expression of one or more genes (such as enzymes) may be tailored to a particular T-cell type, such as a CD4+, CD8+ or regulatory T-cell, or the enzyme may be expressed only when the cell has differentiated to effector memory.

Vector/Kit of Vectors

The present invention also provides a vector, or kit of vectors which comprises one or more construct(s) of the invention or nucleic acid sequence(s) in accordance with the invention. Such a vector or kit of vectors may be used to introduce the nucleic acid sequence(s) or construct(s) into a host cell so that it expresses a CAR or engineered TCR and one or more enzymes which, when secreted or expressed at the cell surface, causes depletion of a molecule extracellular to the engineered cell wherein said molecule is selected from: an amino acid; a nucleotide or nucleoside; or a lipid.

The vector may, for example, be a plasmid or a viral vector, such as a retroviral vector or a lentiviral vector, or a transposon based vector or synthetic mRNA.

The vector may be capable of transfecting or transducing a cell.

The present invention provides a kit of vectors which comprises: (i) a first vector comprising a polynucleotide which encodes one or more enzymes involved in the intracellular synthesis of a molecule; and (ii) a second vector comprising a polynucleotide which encodes a chimeric antigen receptor (CAR) or a transgenic T-cell receptor (TCR).

The molecule may be required by a tumour cell for survival, proliferation, metastasis or chemoresistance. The molecule may be an amino acid; a nucleotide or nucleoside; or a lipid, or a derivative thereof. The molecule may be an amino acid such as an essential amino acid. The molecule may be isoleucine, leucine, lysine, methionine, arginine, phenylalanine, threonine, tryptophan or valine.

The one or more enzyme(s) may be a bacterial enzyme, such as one of the enzymes involved in the biosynthetic pathways shown in FIGS. 5 to 10.

The kit of vectors may also comprise a vector which comprises a polynucleotide which encodes an enzyme which, when secreted or expressed at the cell surface, causes depletion of a molecule extracellular to the cell. The molecule may be required by a tumour cell for survival, proliferation, metastasis or chemoresistance. The molecule may be an amino acid; a nucleotide or nucleoside; or a lipid, or a derivative thereof. The molecule may be an amino acid such as an essential amino acid. The molecule may be isoleucine, leucine, lysine, methionine, arginine, phenylalanine, threonine, tryptophan or valine.

The kit of vectors may also comprise a polynucleotide encoding a dominant negative TGFβ receptor. A dominant negative TGFβ receptor may lack the kinase domain. It may comprise or consist of the sequence shown as SEQ ID No. 20, which is a monomeric version of TGF receptor II

(dn TGFβ RII)
SEQ ID No. 20
TIPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPDLLLVIFQVTG
ISLLPPLGVAISVIIIFYCYRVNRQQKLSS

A dominant-negative TGF-βRII (dnTGF-βRII) has been reported to enhance PSMA targeted CAR-T cell proliferation, cytokine secretion, resistance to exhaustion, long-term in vivo persistence, and the induction of tumour eradication in aggressive human prostate cancer mouse models (Kloss et al (2018) Mol. Ther. 26:1855-1866).

Method for Making a Cell

Engineered cells of the present invention may be produced by introducing DNA or RNA coding for the one or more enzymes as defined herein, to a cell which expresses a CAR to transgenic TCR, by one of many means including transduction with a viral vector, transfection with DNA or RNA.

Alternatively, engineered cells of the present invention may be produced by introducing DNA or RNA coding for a CAR or transgenic TCR and DNA or RNA coding for the one or more enzymes as defined herein, to a cell by one of many means including transduction with a viral vector, transfection with DNA or RNA.

The cell according to the present invention may be made by:
(i) isolation of a cell-containing sample; and
(ii) introducing a nucleic acid construct according to the present invention, a first and second polynucleotide as defined herein, a vector according to the present invention, or a first and second vector as defined herein to the cell.

The cells may then be purified, for example, selected on the basis of expression of the antigen-binding domain of the antigen-binding polypeptide and/or on the basis of expression of said one or more enzymes.

The method for making a cell according to the present invention may be an in vitro method. The method for making a cell according to the present invention may be an ex vivo method.

Suitably, the cell may be from a sample isolated from a subject. Suitably, the cell may be from a sample isolated from any source described above.

Pharmaceutical Composition

The present invention also relates to a pharmaceutical composition containing an engineered cell according to the present invention or a cell obtainable (e.g. obtained) by a method according to the present invention.

The present invention also provides a pharmaceutical composition comprising a nucleic acid construct according to the present invention, a first and second polynucleotide as defined herein, or a vector according to the present invention or a first and second vector as defined herein.

In particular, the invention relates to a pharmaceutical composition containing a cell according to the present invention.

Suitably, the pharmaceutical composition may comprise a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering an engineered cell according to the invention, or an engineered cell obtainable (e.g. obtained) by a method according to the present invention, a nucleic acid construct according to the present invention, a first and second polynucleotide as defined herein, or a vector according to the present invention or a first and second vector as defined herein (for example in a pharmaceutical composition as described above) to a subject.

Suitably, the present invention provides a method for treating and/or preventing a disease which comprises the step of administering the engineered cells of the present invention (for example in a pharmaceutical composition as described above) to a subject.

A method for treating a disease relates to the therapeutic use of the engineered cells of the present invention. In this respect, the engineered cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) introducing a nucleic acid construct according to the present invention, a first and second polynucleotide as defined herein, a vector according to the present invention, or a first and second vector as defined herein to the cell;
(iii) administering the cells from (ii) to a subject.

Suitably, the nucleic acid construct, vector(s) or nucleic acids may be introduced by transduction. Suitably, the nucleic acid construct, vector(s) or nucleic acids may be introduced by transfection.

Suitably, the cell may be autologous. Suitably, the cell may be allogenic.

Where the pharmaceutical composition comprises cells capable of synthesizing a molecule (such as an amino acid) from a precursor, the method may comprise the step of administering the precursor to the subject, before, after or at the same time as the CAR- or TCR-expressing cells are administered to the subject.

The precursor may be citrulline for arginine biosynthesis. In order to enhance citrulline import, the cells may be engineered to express L-type amino acid transporter (LAT1).

The engineered cell may be administered in the form of a pharmaceutical composition. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The present invention provides a cell of the present invention, a nucleic acid construct according to the present invention, a first and second polynucleotide as defined herein, a vector according to the present invention, or a first and second vector as defined herein for use in treating and/or preventing a disease.

The invention also relates to the use of a cell of the present invention, a nucleic acid construct according to the present invention, a first and second polynucleotide as defined herein, a vector according to the present invention, or a first and second vector as defined herein, in the manufacture of a medicament for the treatment and/or prevention of a disease.

In particular, the invention relates to the use of a cell of the present invention in the manufacture of a medicament for the treatment and/or prevention of a disease.

Suitably, the present invention provides a cell of the present invention for use in treating and/or preventing a disease.

The methods may be for the treatment of a cancerous disease. The cancer may be a solid cancer.

The cancer may be a cancer such as neuroblastoma, multiple myeloma, prostate cancer, bladder cancer, breast cancer, colon cancer, endometrial cancer, kidney cancer (renal cell), leukaemia, lung cancer, melanoma, non-Hodgkin lymphoma, pancreatic cancer, and thyroid cancer. Suitably, the cancer may be neuroblastoma. Suitably, the cancer may be multiple myeloma. Suitably, the cancer may be prostate cancer.

The CAR cells of the present invention may be capable of killing target cells, such as cancer cells. The target cell may be recognisable by expression of a TAA, for example the expression of a TAA provided above in Table 2.

The invention will now be further described by way of Examples, which are meant to serve to assist one of ordinary skill in the art in carrying out the invention and are not intended in any way to limit the scope of the invention.

### EXAMPLES

### Example 1—Generation of Methioninase-Expressing Cells

PBMCs isolated from normal subjects activated for 24 hours with anti-CD3 and CD28 antibodies are transduced with a retroviral vector expressing methioninase under the control of the viral LTR promoter. The methioninase is L-methionine gamma-lyase from P. putida (UniProtKB Accession No: P13254).

Transduced T cells are cultured for up to 7 days in RPMI containing 100 u/ml IL2 and methionine levels in the tissue culture medium are measured by ELISA.

### Example 2—Investigating the Effect of Methionine Depletion on Cancer Cells

Transduced T cells produced as described in Example 1 are co-cultured with MDA-MB 231 (a human triple negative breast cancer cell line) at 8:1 and 4:1 T-cell:target ratios for 7 days in a culture medium containing varying concentrations of methionine. Methionine-free media is used as a control. The different media are made by taking commercially available methionine-free media, adding dialysed foetal bovine sera up to a final concentration of 10%, then dividing the media into aliquots and adding varying amounts of methionine.

The effect of methionine production by the T cells is analysed by detecting cellular ATP production. The level of ATP production from metabolically active cells is directly proportional to the numbers of cells present in culture. Proliferation of target cells is measured using FACs staining with the viability dye 7-AAD, and anti-CD3 antibodies are used to gate out the T cells. Numbers of CD3-negative viable cells are then recorded.

In an alternative experiment, the MDA-MB 231 target cells are engineered to express a stably incorporated fluorescent gene. Transduced T cells are co-cultured with target cells as described above in a culture medium containing varying concentrations of methionine. The number of viable target cells is then followed using real time imaging.

### Example 3—Methionine Depletion by T-Cells Expressing Bacterial Methioninase/Methionine Gamma Lyase Enzymes

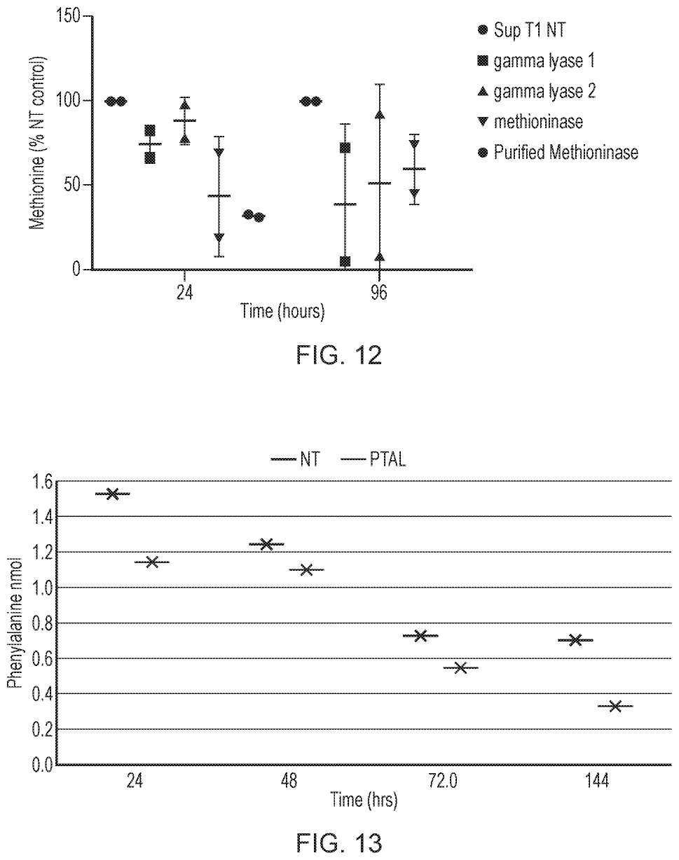

Retroviral constructs encoding genes for Methioninase (Pseudomonas putida: Uniprot P13254), Methionine gamma lyase (Kluyveromyces lactis: Uniprot Q6CKK3), Methionine gamma lyase (Kluyveromyces lactis: Uniprot Q6CKK4), were transduced into the SupT1 T cell line. Expresssion of encoded genes was analysed by expression of V5 Tag expression. Cells were plated at 100,000 cells/ml for 24 or 96 hours and levels of methionine in culture medium was assessed by Methionine assay kit (Biovision). Recombinant methioninase from P. putida was added to culture medium as a control. The results are shown in FIG. 12. Expression of methioninase or either methionine gamma lyase enzyme by the T cells caused depletion of methionine in the culture medium.

### Example 4—Phenylalanine Depletion by T-Cells Expressing Phenylalanine/Tyrosine Ammonia Lyase (PTAL) Enzyme

Retroviral constructs encoding genes for Phenylalanine/tyrosine ammonia lyase (PTAL) were transduced into SupT1 T cell line. Expression of encoded genes was analysed by expression of V5 Tag expression. Cells were plated at 100,000 cells/ml for 24, 48, 72 or 144 hours and levels of phenyalanine in culture medium was assessed by Phenylalanine assay kit (Biovision). The results are shown in FIG. 13. Depletion of phenylalanine in the culture medium was observed for non-transduced cells (NT) presumably due to uptake and use of phenylalanine by the T-cells. However, phenylalanine depletion was increased by the expression of Phenylalanine/tyrosine ammonia lyase (PTAL) by the T cells.

### Example 5—Threonine Depletion by T-Cells Expressing Threonine Dehydrogenase (TDH) or L-Serine Dehydratase (STDH)

Retroviral constructs encoding genes for Threonine dehydrogenase (TDH) or L-serine dehydratase (STDH) were transduced into SupT1 T cell line. TDH is an inactive gene in humans, the sequence used in this study had errors repaired to re-constitute an active enzyme. Expression of encoded genes was analysed by expression of V5 Tag expression. Cells were plated at 100,000 cells/ml for 24, 48, 72 or 144 hours and levels of threonine in culture medium was assessed by Threonine assay kit (Biovision).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which expresses a chimeric antigen receptor (CAR) or engineered cell receptor (TCR) and one or more enzymes which, when secreted or expressed at the cell surface, causes depletion of a molecule extracellular to the cell,
wherein said molecule is selected from: an amino acid, a nucleotide or nucleoside or a lipid.

2. A cell according to claim 1, wherein the molecule is an amino acid.

3. A cell according to claim 2, wherein the amino acid is selected from: isoleucine, leucine, lysine, methionine, arginine, phenylalanine, threonine, tryptophan and valine.

4. A cell according to claim 3, wherein the amino acid is arginine.

5. A cell according to claim 4, which secretes or expresses arginase, arginine deaminase and/or arginine decarboxylase.

6-11. (canceled)

12. A cell according to claim 1, wherein the molecule is a nucleotide or nucleoside.

13-14. (canceled)

15. A cell according to claim 1, wherein the molecule is a lipid.

16. (canceled)

17. A cell according to claim 1, wherein the enzyme(s) convert(s) the molecule into a product which is detrimental to the survival or proliferation of a tumour cell or promotes the proliferation and/or activity of the CAR/TCR-expressing cell.

18. (canceled)

19. A cell according to claim 1, which is engineered to survive in the absence of the molecule in the extracellular environment.

20. A cell according to claim 19, which is engineered to synthesise the molecule or a precursor thereof intracellularly.

21. A cell according to claim 20 which is engineered to synthesise isoleucine, leucine, lysine, methionine, arginine, phenylalanine, threonine, tryptophan or valine.

22. A nucleic acid construct which comprises:
(i) a first polynucleotide which encodes an enzyme which, when secreted or expressed by a cell, causes depletion of a molecule extracellular to the cell,
wherein said molecule is selected from: an amino acid, a nucleotide or nucleoside, or a lipid; and
(ii) a second polynucleotide which encodes a chimeric antigen receptor (CAR) or a transgenic T-cell receptor (TCR).

23-24. (canceled)

25. A vector which comprises a nucleic acid construct according to claim 22.

26. (canceled)

27. A pharmaceutical composition which comprises a plurality of cells according to claim 1.

28. (canceled)

29. A method for treating cancer, which comprises the step of administering a pharmaceutical composition according to claim 27 to a subject in need thereof.

30. A method according to claim 29, which comprises the following steps:
(i) isolation of a cell containing sample,
(ii) introducing to the cell ex vivo:
(a) a first polynucleotide which encodes an enzyme which, when secreted or expressed by a cell, causes depletion of a molecule extracellular to the cell,
wherein said molecule is selected from: an amino acid, a nucleotide or nucleoside, or a lipid, and
(b) a second polynucleotide which encodes a chimeric antigen receptor (CAR) or a transgenic T-cell receptor (TCR); and
(iii) administering the cells from (ii) to a subject.

32. A method according to claim 29 which comprises the following steps:
(i) administering a pharmaceutical composition to the subject, wherein the pharmaceutical composition comprises cells capable of synthesizing the molecule from a precursor, and
(ii) administering the precursor to the subject.

33. A method according to claim 32, wherein the molecule is arginine and the precursor is citrulline.

34. A method according to claim 33, wherein the cells are engineered to express L-type amino acid transporter (LAT1).

35-36. (canceled)

37. A method for making a cell according to claim 1, which comprises the step of introducing into a cell ex vivo:
(a) a first polynucleotide which encodes an enzyme which, when secreted or expressed by a cell, causes depletion of a molecule extracellular to the cell,
wherein said molecule is selected from: an amino acid, a nucleotide or nucleoside, or a lipid, and
(b) a second polynucleotide which encodes a chimeric antigen receptor (CAR) or a transgenic T-cell receptor (TCR).


SEQUENCE LISTING (23 sequences)

SEQ ID NO: 1 - protein, 150 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 2 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 3 - protein, 410 aa, Mycoplasma arginini
MSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW

SEQ ID NO: 4 - protein, 309 aa, Homo sapiens
MVLCIATDDSHSLSCLSLKFGVSLKSCRHLLENAKKHHVEVVGVSFHIGSGCPDPQAYAQSIADARLVFEMGTELGHKMHVLDLGGGFPGTEGAKVRFEEIASVINSALDLYFPEGCGVDIFAELGRYYVTSAFTVAVSIIAKKEVLLDQPGREEENGSTSKTIVYHLDEGVYGIFNSVLFDNICPTPILQKKPSTEQPLYSSSLWGPAVDGCDCVAEGLWLPQLHVGDWLVFDNMGAYTVGMGSPFWGTQACHITYAMSRVAWEALRRQLMAAEQEDDVEGVCKPLSCGWEITDTLCVGPVFTPASIM

SEQ ID NO: 5 - protein, 702 aa, Arabidopsis thaliana
MPALAFVDTPIDTFSSIFTPSSVSTAVVDGSCHWSPSLSSSLYRIDGWGAPYFAANSSGNISVRPHGSNTLPHQDIDLMKVVKKVTDPSGLGLQLPLIVRFPDVLKNRLECLQSAFDYAIQSQGYDSHYQGVYPVKCNQDRFIIEDIVEFGSGFRFGLEAGSKPEILLAMSCLCKGNPEAFLVCNGFKDSEYISLALFGRKLELNTVIVLEQEEELDLVIDLSQKMNVRPVIGLRAKLRTKHSGHFGSTSGEKGKFGLTTVQILRVVRKLSQVGMLDCLQLLHFHIGSQIPSTALLSDGVAEAAQLYCELVRLGAHMKVIDIGGGLGIDYDGSKSGESDLSVAYSLEEYAAAVVASVRFVCDQKSVKHPVICSESGRAIVSHHSVLIFEAVSAGQQHETPTDHQFMLEGYSEEVRGDYENLYGAAMRGDRESCLLYVDQLKQRCVEGFKEGSLGIEQLAGVDGLCEWVIKAIGASDPVLTYHVNLSVFTSIPDFWGIDQLFPIVPIHKLDQRPAARGILSDLTCDSDGKINKFIGGESSLPLHEMDNNGCSGGRYYLGMFLGGAYEEALGGVHNLFGGPSVVRVLQSDGPHGFAVTRAVMGQSSADVLRAMQHEPELMFQTLKHRAEEPRNNNNKACGDKGNDKLVVASCLAKSFNNMPYLSMETSTNALTAAVNNLGVYYCDEAAAGGGGKGKDENWSYFG

SEQ ID NO: 6 - protein, 725 aa, Arabidopsis thaliana
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDSYGVTTGFGATSHRRTKNGVALQKELIRFLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 7 - protein, 398 aa, Pseudomonas putida
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 8 - protein, 328 aa, Homo sapiens
MMSGEPLHVKTPIRDSMALSKMAGTSVYLKMDSAQPSGSFKIRGIGHFCKRWAKQGCAHFVCSSAGNAGMAAAYAARQLGVPATIVVPSTTPALTIERLKNEGATVKVVGELLDEAFELAKALAKNNPGWVYIPPFDDPLIWEGHASIVKELKETLWEKPGAIALSVGGGGLLCGVVQGLQEVGWGDVPVIAMETFGAHSFHAATTAGKLVSLPKITSVAKALGVKTVGAQALKLFQEHPIFSEVISDQEAVAAIEKFVDDEKILVEPACGAALAAVYSHVIQKLQLEGNLRTPLPSLVVIVCGGSNISLAQLRALKEQLGMTNRLPK

SEQ ID NO: 9 - protein, 373 aa, Mus musculus
MLFLGMLKQVVNGTAQSKASSCRKLVLPLKFLGTSQHRIPADANFHSTSISEAEPPRVLITGGLGQLGVGLANLLRKRFGKDNVILSDIRKPPAHVFHSGPFVYANILDYKSLREIVVNHRISWLFHYSALLSAVGEANVSLARDVNITGLHNVLDVAAEYNVRLFVPSTIGAFGPTSPRNPAPDLCIQRPRTIYGVSKVHTELMGEYYYYRYGLDFRCLRYPGIISADSQPGGGTTDYAVQIFHAAAKNGTFECNLEAGTRLPMMYISDCLRATLEVMEAPAERLSMRTYNISAMSFTPEELAQALRKHAPDFQITYCVDPLRQAIAESWPMILDDSNARKDWGWKHDFDLPELVATMLNFHGVSTRVAQVN

SEQ ID NO: 10 - protein, 386 aa, Homo sapiens
MKDCSNGCSAECTGEGGSKEVVGTFKAKDLIVTPATILKEKPDPNNLVFGTVFTDHMLTVEWSSEFGWEKPHIKPLQNLSLHPGSSALHYAVELFEGLKAFRGVDNKIRLFQPNLNMDRMYRSAVRATLPVFDKEELLECIQQLVKLDQEWVPYSTSASLYIRPTFIGTEPSLGVKKPTKALLFVLLSPVGPYFSSGTFNPVSLWANPKYVRAWKGGTGDCKMGGNYGSSLFAQCEAVDNGCQQVLWLYGEDHQITEVGTMNLFLYWINEDGEEELATPPLDGIILPGVTRRCILDLAHQWGEFKVSERYLTMDDLTTALEGNRVREMFGSGTACVVCPVSDILYKGETIHIPTMENGPKLASRILSKLTDIQYGREESDWTIVLS

SEQ ID NO: 11 - protein, 366 aa, Thermoactinomyces intermediusis
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINVADELLGYNRERAMKKVEGIYDKILKVFEIAKRDGIPSYLAADRMAEERIEMMRKTRSTFLQDQRNLINFNNK

SEQ ID NO: 12 - protein, 767 aa, Homo sapiens
MPRQFPKLNISEVDEQVRLLAEKVFAKVLREEDSKDALSLFTVPEDCPIGQKEAKERELQKELAEQKSVETAKRKKSFKMIRSQSLSLQMPPQQDWKGPPAASPAMSPTTPVVTGATSLPTPAPYAMPEFQRVTISGDYCAGITLEDYEQAAKSLAKALMIREKYARLAYHRFPRITSQYLGHPRADTAPPEEGLPDFHPPPLPQEDPYCLDDAPPNLDYLVHMQGGILFVYDNKKMLEHQEPHSLPYPDLETYTVDMSHILALITDGPTKTYCHRRLNFLESKFSLHEMLNEMSEFKELKSNPHRDFYNVRKVDTHIHAAACMNQKHLLRFIKHTYQTEPDRTVAEKRGRKITLRQVFDGLHMDPYDLTVDSLDVHAGRQTFHRFDKFNSKYNPVGASELRDLYLKTENYLGGEYFARMVKEVARELEESKYQYSEPRLSIYGRSPEEWPNLAYWFIQHKVYSPNMRWIIQVPRIYDIFRSKKLLPNFGKMLENIFLPLFKATINPQDHRELHLFLKYVTGFDSVDDESKHSDHMFSDKSPNPDVWTSEQNPPYSYYLYYMYANIMVLNNLRRERGLSTFLFRPHCGEAGSITHLVSAFLTADNISHGLLLKKSPVLQYLYYLAQIPIAMSPLSNNSLFLEYSKNPLREFLHKGLHVSLSTDDPMQFHYTKEALMEEYAIAAQVWKLSTCDLCEIARNSVLQSGLSHQEKQKFLGQNYYKEGPEGNDIRKTNVAQIRMAFRYETLCNELSFLSDAMKSEEITALTN

SEQ ID NO: 13 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 14 - protein, 465 aa, Homo sapiens
MEPSSLELPADTVQRIAAELKCHPTDERVALHLDEEDKLRHFRECFYIPKIQDLPPVDLSLVNKDENAIYFLGNSLGLQPKMVKTYLEEELDKWAKIAAYGHEVGKRPWITGDESIVGLMKDIVGANEKEIALMNALTVNLHLLMLSFFKPTPKRYKILLEAKAFPSDHYAIESQLQLHGLNIEESMRMIKPREGEETLRIEDILEVIEKEGDSIAVILFSGVHFYTGQHFNIPAITKAGQAKGCYVGFDLAHAVGNVELYLHDWGVDFACWCSYKYLNAGAGGIAGAFIHEKHAHTIKPALVGWFGHELSTRFKMDNKLQLIPGVCGFRISNPPILLVCSLHASLEIFKQATMKALRKKSVLLTGYLEYLIKHNYGKDKAATKKPVVNIITPSHVEERGCQLTITFSVPNKDVFQELEKRGVVCDKRNPNGIRVAPVPLYNSFHDVYKFTNLLTSILDSAETKN

SEQ ID NO: 15 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 16 - protein, 266 aa, Homo sapiens [sequence omitted]

SEQ ID NO: 17 - protein, 568 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKAYRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKYGYAPKGSSLVLYSDKKYRNYQFFVDTDWQGGIYASPTIAGSRPGGISAACWAALMHFGENGYVEATKQIIKTARFLKSELENIKGIFVFGNPQLSVIALGSRDFDIYRLSNLMTAKGWNLNQLQFPPSIHFCITLLHARKRVAIQFLKDIRESVTQIMKNPKAKTTGMGAIYGMAQTTVDRNMVAELSSVFLDSLYSTDTVTQGSQMNGSPKPH

SEQ ID NO: 18 - protein, 284 aa, Homo sapiens
MFDKTRLPYVALDVLCVLLAGLPFAILTSRHTPFQRGVFCNDESIKYPYKEDTIPYALLGGIIIPFSIIVIILGETLSVYCNLLHSNSFIRNNYIATIYKAIGTFLFGAAASQSLTDIAKYSIGRLRPHFLDVCDPDWSKINCSDGYIEYYICRGNAERVKEGRLSFYSGHSSFSMYCMLFVALYLQARMKGDWARLLRPTLQFGLVAVSIYVGLSRVSDYKHHWSDVLTGLIQGALVAILVAVYVSDFFKERTSFKERKEEDSHTTLHETPTTGNHYPSNHQP

SEQ ID NO: 19 - protein, 311 aa, Homo sapiens
MQNYKYDKAIVPESKNGGSPALNNNPRRSGSKRVLLICLDLFCLFMAGLPFLIIETSTIKPYHRGFYCNDESIKYPLKTGETINDAVLCAVGIVIAILAIITGEFYRIYYLKKSRSTIQNPYVAALYKQVGCFLFGCAISQSFTDIAKVSIGRLRPHFLSVCNPDFSQINCSEGYIQNYRCRGDDSKVQEARKSFFSGHASFSMYTMLYLVLYLQARFTWRGARLLRPLLQFTLIMMAFYTGLSRVSDHKHHPSDVLAGFAQGALVACCIVFFVSDLFKTKTTLSLPAPAIRKEILSPVDIIDRNNHHNMM

SEQ ID NO: 20 - protein, 177 aa, Artificial Sequence
  dominant negative TGFbeta receptor II
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 21 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 22 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 23 - protein, 7 aa, Artificial Sequence
  consensus Tobacco Etch Virus (TEV) cleavage site
ENLYFQS